SEQUENCE LISTING (10 sequences)

SEQ ID NO: 1 - protein, 633 aa, Artificial Sequence
  amino acid sequence of Cry2Ai protein (NCBI GenBank ACV97158.1)
MNNVLNSGRNITCHAHNVVAHDPFSFEHKSLNTIEKEWKEWKRTDHSLYVAPIVGTVGSFLLKKVGSLVGKRILSELQNLIFPSGSIDLMQEILRATEQFINQRLNADTLGRVNAELAGLQANVAEFNRQVDNFLNPNQNPVPLAIIDSVNTLQQLFLSRLPQFQIQGYQLLLLPLFAQAANLHLSFIRDVILNADEWGISAATVRTYRDHLRNFTRDYSNYCINTYQTAFRGLNTRLHDMLEFRTYMFLNVFEYVSIWSLFKYQSLMVSSGANLYASGSGPQQTQSFTAQNWPFLYSLFQVNSNYILSGISGTRLSITFPNIGGLPGSTTTHSLNSARVNYSGGVSSGLIGATNLNHNFNCSTVLPPLSTPFVRSWLDSGTDREGVATSTNWQTESFQTTLSLRCGAFSARGNSNYFPDYFIRNISGVPLVIRNEDLTRPLHYNQIRNIESPSGTPGGARAYLVSVHNRKNNIYAANEYGTMIHLAPEDYTGFTISPIHATQVNNQTRTFISEKFGNQGDSLRFEQSNTTARYTLRGNGNSYNLYLRASSIGNSTIRVTINGRAYTVSNVNTTTNNDGVNDNGARFSDINIGNIVASDNTNVTLDINVTLNSGTPFDLMNIMFVPTNLPPLY

SEQ ID NO: 2 - DNA, 1944 bp, Artificial Sequence
  201D1
tctagaccatggggatccatgaataatgttcttaactctggaaggaatattacctgccatgcacataatgttgttgcacatgacccattcagttttgaacataagagtctaaacactatagaaaaagagtggaaggagtggaaaaggacagatcattctctatacgtagctccaatcgtggggactgtgggttcttttctattaaagaaggtaggttccttagtgggtaagagaatactgtctgaacttcaaaatctgatcttcccatctggtagtattgacctgatgcaggaaatacttcgtgctactgagcaattcattaaccaacgtttgaatgctgatacacttggtagggtaaatgccgaactcgctggattgcaggccaatgttgctgagtttaacaggcaagtggataactttttaaaccctaaccaaaatccagtcccacttgctattattgattccgtcaacacattacagcagttattcctttcacgactgcctcaattccaaatccaggggtatcaactcctactattgccgctattcgctcaagccgctaacttgcacctgagttttatcagagatgttattttgaatgcagacgaatgggggatttcagccgcaactgtgagaacttatagagatcatctacgtaactttacaagagattactctaactactgtattaacacatatcaaacagcttttagaggcttgaacactaggcttcacgatatgcttgagtttaggacatacatgtttttgaacgttttcgagtatgttagtatttggtcactatttaagtatcaatcccttatggtatcctctggagcaaacctatacgcctccggttcaggcccccaacaaacccaatcctttacagcacagaactggccatttttgtattcattgtttcaagtgaactctaattacattctctcaggtattagcggtacaagactttctattacattccccaatataggaggactcccaggttcaacaacaactcattcattgaacagtgcaagggttaactattctggcggagtcagttcaggtcttattggcgcaacaaatcttaatcacaatttcaactgttcaaccgtgttaccaccgttaagcacaccattcgtaaggagttggttagactcagggacagatagagaaggagtcgcaacatcaacgaattggcagactgagtctttccaaacaactctttcacttaggtgtggagcctttagcgctagggggaacagcaactattttcctgactactttataagaaacatatcaggtgtcccattggttattaggaacgaggatttaacaaggcccctacattacaaccagataagaaatattgagtcaccgtccggtactccaggtggtgctagggcttatttagtgagcgtgcataataggaagaataacatttacgccgcaaacgaatatggaactatgatacatcttgctccggaggattataccgggtttaccattagtccaatccacgcaacacaagtgaataatcagacaaggacttttatatctgaaaaattcggcaatcaaggagattctcttaggtttgagcaatctaacacgaccgctcgttacacccttcgtggcaacggaaatagctacaatttgtatctccgtgcttcatctatcgggaactcaactattcgtgttacaatcaatggtagagcatacaccgttagtaatgtaaatacaactactaacaatgatggcgtaaatgataatggagcaaggttcagcgacataaacatagggaacattgtcgcaagtgacaacactaacgttaccttggatataaatgttacattaaactccggcactccttttgacttgatgaatattatgttcgtaccaactaatctcccaccgctgtatcccgggtgagaattcaagcttgagctc

SEQ ID NO: 3 - DNA, 1944 bp, Artificial Sequence
  201D2
tctagaccatggggatccatgaataatgttttgaattctggaagaaatattacttgtcatgctcataatgttgttgctcatgatcctttttcttttgaacataagtctttgaatactattgaaaaggaatggaaggaatggaagagaactgatcattctttgtatgttgctcctattgttggaactgttggatcttttttgttgaagaaggttggatctttggttggaaagagaattttgtctgaattgcaaaatctgatcttcccatctggtagtattgacctgatgcaggaaatacttcgtgctactgagcaattcattaaccaacgtttgaatgctgatacacttggtagggtaaatgccgaactcgctggattgcaggccaatgttgctgagtttaacaggcaagtggataactttttaaaccctaaccaaaatccagtcccacttgctattattgattctgtcaacacattacagcagttattcctttcaagactgcctcaattccaaatccaggggtatcaactcctactattgccgctattcgctcaagctgctaacttgcacctgagttttatcagagatgttattttgaatgcagacgaatgggggatttcagctgcaactgtgagaacttatagagatcatctacgtaactttacaagagattactctaactactgtattaacacatatcaaacagcttttagaggattgaacactaggcttcacgatatgcttgagtttaggacatacatgtttttgaacgttttcgagtatgttagtatttggtcactatttaagtatcaatctcttatggtatcttctggagcaaacctatacgcttctggttcaggacctcaacaaacccaatcttttacagcacagaactggccatttttgtattcattgtttcaagtgaactctaattacattctctcaggtattagcggtacaagactttctattacattccctaatataggaggactcccaggttcaacaacaactcattcattgaacagtgcaagggttaactattctggaggagtcagttcaggtcttattggagcaacaaatcttaatcacaatttcaactgttcaaccgtgttaccaccgttaagcacaccattcgtaaggagttggttagactcagggacagatagagaaggagtcgcaacatcaacgaattggcagactgagtctttccaaacaactctttcacttaggtgtggagcttttagcgctagggggaacagcaactattttcctgactactttataagaaacatatcaggtgtcccattggttattaggaacgaggatttaacaaggcctctacattacaaccagataagaaatattgagtcaccgtctggtactccaggtggtgctagggcttatttagtgagcgtgcataataggaagaataacatttacgctgcaaacgaatatggaactatgatacatcttgctccggaggattataccgggtttaccattagtccaatccacgcaacacaagtgaataatcagacaaggacttttatatctgaaaaattcggcaatcaaggagattctcttaggtttgagcaatctaacacgaccgctcgttacacccttcgtggaaatggaaattcttataatttgtatttgagagcttcttctattggaaattctactattagagttactattaatggaagagcttatactgtttctaatgttaatactactactaataatgatggagttaatgataatggagctagattttctgatattaatattggaaatattgttgcttctgataatactaatgttactttggatattaatgttactttgaattctggaactccttttgatttgatgaatattatgtttgttcctactaatttgcctcctttgtatcctggataagaattcaagcttgagctc

SEQ ID NO: 4 - DNA, 1944 bp, Artificial Sequence
  201D3
tctagaccatggggatccatgaataatgttttaaactctggtcgtaacattacatgtcatgcacataacgtagtagcacatgatccattctctttcgaacataaatctttaaacacaattgaaaaagaatggaaagaatggaaacgtacagatcattctttatacgtagcaccaattgtaggtacagtaggttctttcttattaaaaaaagtaggttctttagtaggtaaacgtattttatctgaattacaaaacttaattttcccatctggtagtattgacctgatgcaggaaatacttcgtgctactgagcaattcattaaccaacgtttgaatgctgatacacttggtagggtaaatgccgaactcgctggattgcaggccaatgttgctgagtttaaccgtcaagtagataactttttaaaccctaaccaaaatccagtaccacttgctattattgattctgtaaacacattacaacaattattcctttcacgtttacctcaattccaaatccaaggttatcaattactactattaccactattcgctcaagcagctaacttacacttaagttttatccgtgatgttattttaaatgcagacgaatggggtatttcagcagcaactgtacgtacttatcgtgatcatctacgtaactttacacgtgattactctaactactgtattaacacatatcaaacagcttttcgtggcttaaacactcgtcttcacgatatgcttgagtttcgtacatacatgtttttaaacgttttcgagtatgttagtatttggtcactatttaagtatcaatctcttatggtatcttctggagcaaacctatacgcatctggttcaggcccacaacaaacacaatcttttacagcacaaaactggccatttttatattcattatttcaagtaaactctaattacattttatcaggtattagcggtacacgtctttctattacattcccaaatattggaggattaccaggttcaacaacaactcattcattaaacagtgcacgtgttaactattctggcggagtaagttcaggtcttattggcgcaacaaatcttaatcacaatttcaactgttcaacagtattaccaccattaagcacaccattcgtacgtagttggttagactcaggtacagatcgtgaaggagtagcaacatcaacgaattggcaaactgagtctttccaaacaactctttcacttcgttgtggagcatttagcgctcgtggtaacagcaactattttcctgactactttattcgtaacatttcaggtgtaccattagttattcgtaacgaggatttaacacgtccactacattacaaccaaattcgtaatattgagtcaccatctggtactccaggtggtgctcgtgcttatttagtaagcgtacataatcgtaagaataacatttacgcagcaaacgaatatggaactatgatacatcttgctccggaggattataccgggtttaccattagtccaatccacgcaacacaagtgaataatcagacaaggacttttatatctgaaaaattcggcaatcaaggagattctcttaggtttgagcaatctaacacgaccgctcgttacacccttcgtggtaacggtaactcttacaacttatacttacgtgcatcttctattggtaactctacaattcgtgtaacaattaacggtcgtgcatacacagtatctaacgtaaacacaacaacaaacaacgatggtgtaaacgataacggtgcacgtttctctgatattaacattggtaacattgtagcatctgataacacaaacgtaacattagatattaacgtaacattaaactctggtacaccattcgatttaatgaacattatgttcgtaccaacaaacttaccaccattatacccaggttaagaattcaagcttgagctc

SEQ ID NO: 5 - DNA, 1944 bp, Artificial Sequence
  201D4
tctagaccatggggatccatgaataatgttctgaacagcggccgcaacatcacgtgccatgcccataacgtcgtcgcccatgacccgtttagctttgaacataaaagcctgaacacgatcgaaaaagaatggaaagaatggaaacgcacggaccatagcctgtatgtcgccccgatcgtcggcacggtcggcagctttctgctgaaaaaagtcggcagcctggtcggcaaacgcatcctgagcgaacttcaaaatctgatcttcccatctggtagtattgacctgatgcaggaaatacttcgtgctactgagcaattcattaaccaacgtttgaatgctgatacacttggtagggtaaatgccgaactcgctggattgcaggccaatgttgctgagtttaacaggcaagtggataactttttaaaccctaaccaaaatccggtcccgcttgctattattgattccgtcaacacattacagcagttattcctttcacgcctgcctcaattccaaatccaggggtatcaactcctgctgttgccgctgttcgctcaagccgctaacttgcacctgagctttatcagagatgttattttgaatgcagacgaatgggggatttcagccgcaacggtgagaacgtatagagatcatctgcgcaactttacaagagattactctaactactgtattaacacatatcaaacagcttttagaggcttgaacacgaggcttcacgatatgcttgagtttaggacatacatgtttttgaacgttttcgagtatgttagcatttggtcactgtttaagtatcaatcccttatggtatcctctggagcaaacctgtacgcctccggttcaggcccgcaacaaacccaatcctttacagcacagaactggccgtttttgtattcattgtttcaagtgaactctaattacattctctcaggtattagcggtacaagactttctattacattcccgaatatcggaggactcccgggttcaacaacaacgcattcattgaacagcgcaagggttaactattctggcggagtcagctcaggtcttattggcgcaacaaatcttaatcacaatttcaactgttcaaccgtgttaccgccgttaagcacaccgttcgtaaggagctggttagactcagggacagatagagaaggagtcgcaacatcaacgaattggcagacggagtctttccaaacaacgctttcacttaggtgtggagcctttagcgctagggggaacagcaactattttcctgactactttatcagaaacatctcaggtgtcccgttggttattaggaacgaggatttaacaaggccgctgcattacaaccagatcagaaatattgagtcaccgtccggtacgccgggtggtgctagggcttatttagtgagcgtgcataataggaagaataacatttacgccgcaaacgaatatggaactatgatacatcttgctccggaggattataccgggtttaccattagtccaatccacgcaacacaagtgaataatcagacaaggacttttatatctgaaaaattcggcaatcaaggagattctcttaggtttgagcaatctaacacgaccgctcgttacacccttcgtggcaacggcaacagctataacctgtatctgcgcgccagcagcatcggcaacagcacgatccgcgtcacgatcaacggccgcgcctatacggtcagcaacgtcaacacgacgacgaacaacgacggcgtcaacgacaacggcgcccgctttagcgacatcaacatcggcaacatcgtcgccagcgacaacacgaacgtcacgctggacatcaacgtcacgctgaacagcggcacgccgtttgacctgatgaacatcatgtttgtcccgacgaacctgccgccgctgtatccgggctaagaattcaagcttgagctc

SEQ ID NO: 6 - DNA, 1944 bp, Artificial Sequence
  201D5
tctagaccatggggatccatgaataatgttctgaactctggtcgtaacatcacttgtcacgctcacaacgtagtagctcacgatccgttctctttcgaacacaaatctctgaacactatcgaaaaagaatggaaagaatggaaacgtactgatcactctctgtacgtagctccgatcgtaggtactgtaggttctttcctgctgaaaaaagtaggttctctggtaggtaaacgtatcctgtctgaactgcaaaatctgatcttcccatctggtagtattgacctgatgcaggaaatacttcgtgctactgagcaattcattaaccaacgtttgaatgctgatacacttggtagggtaaatgccgaactcgctggattgcaggccaatgttgctgagtttaaccgtcaagtggataactttttaaaccctaaccaaaatccggtaccgcttgctattattgattctgtaaacacattacagcagttattcctttcacgtctgcctcaattccaaatccagggttatcaactcctgctgttgccgctgttcgctcaagccgctaacttgcacctgagttttatcagagatgttattttgaatgcagacgaatggggtatttcagccgcaactgtgagaacttatagagatcatctgcgtaactttacaagagattactctaactactgtattaacacatatcaaacagcttttagaggcttgaacactcgtcttcacgatatgcttgagtttcgtacatacatgtttttgaacgttttcgagtatgttagtatttggtcactgtttaagtatcaatctcttatggtatcttctggagcaaacctgtacgcctctggttcaggcccccaacaaacccaatcttttacagcacagaactggccgtttttgtattcattgtttcaagtgaactctaattacattctctcaggtattagcggtacaagactttctattacattccccaatatcggaggactcccgggttcaacaacaactcattcattgaacagtgcacgtgttaactattctggcggagtaagttcaggtcttattggcgcaacaaatcttaatcacaatttcaactgttcaaccgtgttaccgccgttaagcacaccgttcgtacgtagttggttagactcaggtacagatagagaaggagtagcaacatcaactaattggcagactgagtctttccaaacaactctttcacttcgttgtggagcctttagcgctcgtggtaacagcaactattttcctgactactttatcagaaacatctcaggtgtaccgttggttattcgtaacgaggatttaacacgtcccctgcattacaaccagatcagaaatattgagtcaccgtctggtactccgggtggtgctcgtgcttatttagtgagcgtgcataatcgtaagaataacatttacgccgcaaacgaatatggaactatgatccatcttgctccggaggattataccgggtttaccattagtccaatccacgcaacacaagtgaataatcagacaaggacttttatatctgaaaaattcggcaatcaaggagattctcttaggtttgagcaatctaacacgaccgctcgttacacccttcgtggtaacggtaactcttacaacctgtacctgcgtgcttcttctatcggtaactctactatccgtgtaactatcaacggtcgtgcttacactgtatctaacgtaaacactactactaacaacgatggtgtaaacgataacggtgctcgtttctctgatatcaacatcggtaacatcgtagcttctgataacactaacgtaactctggatatcaacgtaactctgaactctggtactccgttcgatctgatgaacatcatgttcgtaccgactaacctgccgccgctgtacccgggttaagaattcaagcttgagctc

SEQ ID NO: 7 - DNA, 20 bp, Artificial Sequence
  is a forward primer sequence for amplification of 201D1 DNA sequence (SEQ ID NO 2)
ctccaatcgtggggactgtg

SEQ ID NO: 8 - DNA, 20 bp, Artificial Sequence
  is a reverse primer sequence for amplification of 201D1 DNA sequence (SEQ ID NO 2)
cccgatagatgaagcacgga

SEQ ID NO: 9 - DNA, 20 bp, Artificial Sequence
  is a forward primer sequence for amplification for nptii dna gene
acgaggaagcggtcagccca

SEQ ID NO: 10 - DNA, 20 bp, Artificial Sequence
  is a reverse primer sequence for amplification for nptII DNA gene
ttgcacgcaggttctccggc

(19) United States
(12) Patent Application Publication
(10) Pub. No.: US 2022/0275031 A1
(43) Pub. Date: Sep. 1, 2022

(54) SYNTHETIC NUCLEOTIDE SEQUENCES ENCODING INSECTICIDAL CRYSTAL PROTEIN AND USES THEREOF

(71) Applicant: DCM SHRIRAM LIMITED, AEROCITY NEW DELHI (IN)

(72) Inventors: DWARKESH SINGH PARIHAR, TELANGANA HYDERABAD (IN); PARESH VERMA, TELANGANA HYDERABAD (IN)

(21) Appl. No.: 17/625,904

(22) PCT Filed: Jul. 28, 2020
(86) PCT No.: PCT/IN2020/050660

§ 371 Date: Jan. 10, 2022

(30) Foreign Application Priority Data
Jul. 30, 2019 (IN) 201911030820

(51) Int. Cl. C07K 14/325; C12N 1/20; A01N 63/23; C12N 15/82

(52) U.S. Cl. CPC C07K 14/325; C12N 1/20; A01N 63/23; C12N 15/8286; C12N 2800/22; C12R 2001/075

(57) ABSTRACT

The present disclosure provides codon optimized synthetic nucleotide sequences encoding Bacillus thuringiensis (Bt) insecticidal crystal protein having insecticidal activity against insect pests. The present disclosure also relates to expression of these sequences in plants. The disclosure further provides a DNA construct, a vector, and a host cell comprising the codon optimized synthetic nucleotide sequences of the invention. Also it provides use of the codon optimized synthetic nucleotide sequences for production of insect resistant transgenic plants, insect resistant transgenic plant comprising the said sequence and a composition comprising Bacillus thuringiensis comprising the codon optimized synthetic nucleotide sequence of the present invention.

### REFERENCE TO SEQUENCE LISTING SUBMITTED ELECTRONICALLY

An official copy of the Sequence Listing a file named “PD034766IN-SC sequence listing.txt” created on 30 Jul. 2019, having a size of 11 kb filed electronically concurrently with the Specification is part of the Specification.

### FIELD

The present disclosure is related to codon optimized synthetic nucleotide sequences encoding Bacillus thuringiensis (Bt) insecticidal crystal protein having insecticidal activity against insect pests. The present disclosure also relates to expression of these sequences in plants.

### BACKGROUND

Insect pests are a major factor in the loss of the world's agricultural crops and said to be responsible for destroying one fifth of the world's total crop production annually. In the process of artificially selecting suitable crops for human consumption, highly susceptible plants for infestations by insects are also selected that ultimately reduced its economic value and increased production cost.

Traditionally, the insect pests are controlled by application of chemical and/or biological pesticides. There are certain concerns of using chemical pesticides due to the environmental hazards associated with the production and use of chemical pesticides. Because of such concerns, regulators have banned or limited the use of some of the more hazardous pesticides.

Further, it is well known fact that insect pests are capable of evolving overtime as a process of natural selection that can adapt to new situations, for example, overcome the effect of toxic materials or bypass natural or artificial plant resistant, which further adds to the problem.

Biological pesticide is an environmentally and commercially acceptable alternative to the chemical pesticides. It presents a lower risk of pollution and environmental hazards, and provides greater target specificity than the traditional broad-spectrum chemical insecticides.

Certain species of microorganisms of the genus Bacillus for example Bacillus thuringiensis (B.t.) are known to possess insecticidal activity against a broad range of insect pests. The insecticidal activity appears to be concentrated in parasporal crystalline protein inclusions bodies, although insecticidal proteins have also been isolated from the vegetative growth stage of Bacillus thuringiensis.

Expression of Bacillus thuringiensis (Bt) insecticidal crystal (cry) protein genes in plants is known in the art however it was found that it is extremely difficult to express the native Bt gene in plants. Attempts have been made to express Bt cry protein gene in plants in combinations with various promoters functional in plants. However, only low levels of protein have been obtained in transgenic plants.

One of the reasons for low level expression of the Bt cry gene in plant is high A/T content in Bt DNA sequence than plant genes in which G/C ratio is higher than A/T. The overall value of A/T for bacterial genes is 60-70% and plant genes with 40-50%. As a consequence, GC ratio in cry genes codon usage is significantly insufficient to express at optimal level. Moreover, the A/T rich region may also contain transcriptional termination sites (AATAAA polyadenylation), mRNA instability motif (ATTTA) and cryptic mRNA splicing sites. It has been observed that the codon usage of a native Bt cry gene is significantly different from that of a plant gene. As a result, the mRNA from this gene may not be efficiently utilized. Codon usage might influence the expression of genes at the level of translation or transcription or mRNA processing. To optimize an insecticidal gene for expression in plants, attempts have been made to alter the gene to resemble, as much as possible, genes naturally contained within the host plant to be transformed.

However, development of crop plant varieties expressing high/optimum level of Bt cry protein conferring resistance to certain insect pests is still a major problem in agricultural field. Increased expression of insect-control protein genes has been critical to the development of genetically improved plants with agronomically acceptable levels of insect resistance. Various attempts to control or prevent insect infestation of crop plants have been made, yet certain insect pests remain to be a significant problem in agriculture. Therefore, there remains a need for insect resistant transgenic crop plants with desired expression levels of insecticidal proteins in the transgenic plants.

The present invention provides herein a solution to the existing problem of insect pest infestation by providing plant codon optimized synthetic DNA sequence encoding Bt Cry2Ai protein having insecticidal activity against insect pests.

### BRIEF SUMMARY

Disclosed herein are codon optimised synthetic nucleotide sequences encoding B. thuringiensis protein having insecticidal activity against insect pests. The disclosure is drawn to methods for enhancing expression of heterologous genes in plant cells. A gene or coding region of cry2Ai gene is constructed to provide a plant specific preferred codon sequence. In this manner, codon usage for a Cry2Ai protein is altered for expression in a plant. Such plant optimized coding sequences can be operably linked to promoter capable of directing expression of the coding sequences in a plant cell. Transformed host cells and transgenic plants comprising the codon optimised B. thuringiensis synthetic nucleotide sequences are also aspects of the present disclosure.

One of the objects of the present disclosure is to provide codon optimized synthetic nucleotide sequences encoding insecticidal protein, wherein the nucleotide sequences have been optimized for expression in plants.

It is another object of the present disclosure to provide codon optimized nucleotide sequences encoding insecticidal Bt protein to maximize the expression of Bt proteins in a plant, preferably in a plant selected from a group consisting of eggplant, cotton, rice, tomato, wheat, corn, sorghum, oat, millet, legume, cabbage, cauliflower, broccoli, Brassica sp., beans, pea, pigeon-pea, potato, pepper, cucurbit, lettuce, sweet potato canola, soybean, alfalfa, peanuts, and sunflower.

According to the present disclosure, the inventors have synthesized Bt insecticidal Cry2Ai crystal protein genes in which the codon usage has been altered in order to increase expression in plant. However, rather than alter the codon usage to resemble plant gene in terms of overall codon distribution, the inventors have optimized the codon usage by using the codons which are most preferred in plants in the synthesis of the nucleotide sequences of the disclosure. The optimized plant preferred codon usage is effective for expression of high level of the Bt insecticidal protein in dicot plants such as cotton, eggplant and tomato; in monocots such as rice and in legumes such as chickpea and pigeon pea.

The codon optimized synthetic nucleotide sequences of the present disclosure have been derived from the Bacillus thuringiensis Cry2Ai protein having amino acid sequence as set forth in SEQ ID NO: 1 (NCBI GenBank: ACV97158.1). The protein having amino acid sequence as set forth in SEQ ID NO: 1 is active against various lepidopteran insects, including Helicoverpa armigera—the cotton bollworm and corn earworm, Cnaphalocrocis medinalis—the rice leaffolder, and Scirpophaga incertulas—the rice yellow stem borer and Pectinophora gossypiella.

While the present disclosure has been exemplified by the synthesis of codon optimized Bt cry2Ai nucleotides for expression in plants. It is recognized that the codon optimized Bt cry2Ai nucleotides can be utilized to optimize expression of the protein in plants such as cotton, eggplant, tomato, rice, and maize.

Accordingly one aspect of present disclosure is to provide a codon optimized synthetic nucleotide sequence encoding protein having amino acid sequence as set forth in SEQ ID NO: 1, wherein said nucleotide sequence is selected from a group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and (b).

Another aspect of the present invention is to provide a nucleic acid molecule comprising a codon optimized sequence for expression in a plant selected from the group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and b).

Another aspect of the present disclosure is to provide a codon optimized synthetic nucleotide sequence encoding protein having the amino acid sequence as set forth in SEQ ID NO: 1, wherein said nucleotide sequence is
a. as set forth in SEQ ID NO: 2, or a nucleotide sequence complementary thereto; or
b. a nucleotide sequence which specifically hybridizes to at least 10 nucleotides of the nucleotide sequence as set forth in SEQ ID NO: 2 from nucleotide position 262 to 402 and/or 1471 to 1631 or a nucleotide sequence complementary thereto

Another aspect of the present disclosure is to provide a recombinant DNA comprising the codon optimized synthetic nucleotide sequence as disclosed herein, wherein the nucleotide sequence is operably linked to a heterologous regulatory element.

Another aspect of the present disclosure is to provide a DNA construct for expression of an insecticidal protein of interest comprising a 5′ non-translated sequence, a coding sequence encoding an insecticidal Cry2Ai protein comprising the amino acid sequence of SEQ ID NO: 1 or an insecticidal portion thereof, and a 3′ non-translated region, wherein said 5′ non-translated sequence comprises a promoter functional in a plant cell, said coding sequence is a codon optimized synthetic nucleotide sequence as disclosed herein, and wherein said 3′ non-translated sequence comprises a transcription termination sequence and a polyadenylation signal.

Another aspect of the present disclosure is to provide a plasmid vector comprising the recombinant DNA disclosed herein, or the DNA construct as disclosed herein.

Another aspect of the present disclosure is to provide a host cell comprising the codon optimized synthetic nucleotide sequence as disclosed herein.

Another aspect of the present disclosure is to provide a method for conferring an insect resistance in a plant comprising
(a) inserting into a plant cell a codon optimized synthetic nucleotide sequence as disclosed herein, wherein the nucleotide sequence is operably linked to a (i) promoter functional in a plant cell and (ii) a terminator;
(b) obtaining a transformed plant cell from the plant cell of step (a), wherein said transformed plant cell comprises the said codon optimized synthetic nucleotide sequence as disclosed herein; and
(c) generating a transgenic plant from said transformed plant cell of step (b), wherein said transgenic plant comprises the said codon optimized synthetic nucleotide sequence as disclosed herein.

Another aspect of the present disclosure is to provide a transgenic plant comprising the codon optimized synthetic nucleotide sequence as disclosed herein.

Another aspect of the present disclosure is to provide a composition comprising Bacillus thuringiensis comprising the codon optimized synthetic nucleotide sequence as disclosed herein encoding Cry2Ai protein having amino acid sequence as set forth in SEQ ID NO: 1.

Another aspect of the present disclosure is to provide a method of controlling insect infestation in a crop plant and providing insect resistance management, wherein said method comprising contacting said crop plant with an insecticidally effective amount of the composition as disclosed herein.

Yet another aspect of the present disclosure is use of the codon optimized synthetic nucleotide sequence, the DNA construct or the plasmid as disclosed herein for production of insect resistant transgenic plants.

Yet another aspect of the present disclosure is use of the codon optimized synthetic nucleotide sequence as disclosed herein for production of insecticidal composition, wherein the composition comprises Bacillus thuringiensis cells comprising the said nucleotide sequences.

### BRIEF DESCRIPTION OF THE ACCOMPANYING FIGURES

FIG. 1 shows T-DNA construct map of pGreen0029-CaMV35S-201D1.

FIG. 2 shows genetic transformation and regeneration of transgenic cotton plants.

### BRIEF DESCRIPTION OF THE SEQUENCES

SEQ ID NO: 1 is amino acid sequence of Cry2Ai protein (NCBI GenBank: ACV97158.1).

SEQ ID NO: 2 is a codon optimized synthetic cry2Ai nucleotide sequence (201D1) encoding the Cry2Ai protein (SEQ ID NO: 1).

SEQ ID NO: 3 is a codon optimized synthetic cry2Ai nucleotide sequence (201D2) encoding the Cry2Ai protein (SEQ ID NO: 1).

SEQ ID NO: 4 is a codon optimized synthetic cry2Ai nucleotide sequence (201D3) encoding the Cry2Ai protein (SEQ ID NO: 1).

SEQ ID NO: 5 is a codon optimized synthetic cry2Ai nucleotide sequence (201D4) encoding the Cry2Ai protein (SEQ ID NO: 1).

SEQ ID NO: 6 is a of codon optimized synthetic cry2Ai nucleotide sequence (201D5) encoding the Cry2Ai protein (SEQ ID NO: 1).

SEQ ID NO: 7 is a forward primer sequence for amplification of 201D1 DNA sequence

(SEQ ID NO: 2).

SEQ ID NO: 8 is a reverse primer sequence for amplification of 201D1 DNA sequence

(SEQ ID NO: 2).

SEQ ID NO: 9 is a forward primer sequence for amplification for nptII DNA gene.

SEQ ID NO: 10 is a reverse primer sequence for amplification for nptII DNA gene.

### DETAILED DESCRIPTION

The detailed description provided herein is to assist person skilled in the art in practicing the present invention and should not be construed to unduly limit scope of the invention as modifications and variations in the embodiments discussed herein may be made by those of person skilled in the art without departing from the spirit or scope of the invention. The present inventions will be described more fully hereinafter with reference to the accompanying drawings and/or sequence listing, in which some, but not all embodiments of the inventions are shown and/or described. The invention may be embodied in many different forms and should not be construed as limited to the embodiments set forth herein.

Although specific terms are employed herein, they are used in a generic and descriptive sense only and not for purposes of limitation. The following definitions are provided to facilitate understanding of the embodiments.

It must be noted that, as used in the specification and the appended claims, the singular forms “a,” “an” and “the” are used herein to refer to one or more than one (i.e., to at least one) of the grammatical object of the article unless the context clearly dictates otherwise. Thus, for example, reference to “a probe” means that more than one such probe can be present in the composition. Similarly, reference to “an element” means one or more element.

Throughout the specification the word “comprises” or “comprising,” will be understood to imply the inclusion of a stated element, integer or step, or group of elements, integers or steps, but not the exclusion of any other element, integer or step, or group of elements, integers or steps.

Units, prefixes, and symbols may be denoted in their SI accepted form. Unless otherwise indicated, nucleic acids are written left to right in 5′ to 3′ orientation and amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The above-defined terms are more fully defined by reference to the specification as a whole.

The term “nucleic acid” typically refers to large polynucleotides. The term “nucleic acid” and “nucleotide sequence” are used interchangeably herein. It includes reference to a deoxyribonucleotide or ribonucleotide polymer in either single- or double-stranded form, and unless otherwise limited, encompasses known analogues (e.g., peptide nucleic acids) having the essential nature of natural nucleotides in that they hybridize to single-stranded nucleic acids in a manner similar to that of naturally occurring nucleotides. Nucleotides are the subunit that is polymerized (connected into a long chain) to make nucleic acids (DNA and RNA). Nucleotides consist of three smaller components a ribose sugar, a nitrogenous base, and phosphate group(s).

A “polynucleotide” means a single strand or parallel and anti-parallel strands of a nucleic acid. Thus, a polynucleotide may be either a single-stranded or a double-stranded nucleic acid.

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The terms “codon optimized synthetic nucleotide sequences”, are the non-genomic nucleotide sequences and are used interchangeably herein to refer a synthetic nucleotide sequences or nucleic acid molecule that has one or more change in the nucleotide sequence compared to a native or genomic nucleotide sequence. In some embodiments the change to a native or genomic nucleic acid molecule includes but is not limited to changes in the nucleic acid sequence due to codon optimization of the nucleic acid sequence for expression in a particular organism, for example a plant, the degeneracy of the genetic code, changes in the nucleic acid sequence to introduce at least one amino acid substitution, insertion, deletion and/or addition compared to the native or genomic sequence, changes in the nucleic acid sequence to introduce restriction enzyme sites, removal of one or more intron associated with the genomic nucleic acid sequence, insertion of one or more heterologous introns, deletion of one or more upstream or downstream regulatory regions associated with the genomic nucleic acid sequence, insertion of one or more heterologous upstream or downstream regulatory regions, deletion of the 5′ and/or 3′ un-translated region associated with the genomic nucleic acid sequence, insertion of a heterologous 5′ and/or 3′ un-translated region, and modification of a polyadenylation site.

In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

In some embodiments the non-genomic nucleic acid molecule is a cDNA. In some embodiments the non-genomic nucleic acid molecule is a synthetic nucleotide sequence. Codon-optimized nucleotide sequences may be prepared for any organism of interest using methods known in the art for example, Murray et al. (1989) Nucleic Acids Res. 17:477-498. Optimized nucleotide sequences find use in increasing expression of a pesticidal protein in a plant, for example monocot and dicot plants such as, rice, tomato, and cotton plant.

The newly designed cry2Ai DNA sequences disclosed herein are referred as “codon optimized synthetic cry2Ai nucleotide sequences”.

The terms “DNA construct”, “nucleotide constructs”, and “DNA expression cassette” are used interchangeably herein and is not intended to limit the embodiments to nucleotide constructs comprising DNA. Those of ordinary skill in the art will recognize that nucleotide constructs particularly polynucleotides and oligonucleotides composed of ribonucleotides and combinations of ribonucleotides and deoxyribonucleotides may also be employed in the methods disclosed herein.

A “recombinant” nucleic acid molecule or DNA or polynucleotide is used herein to refer to a nucleic acid molecule or DNA polynucleotide that has been altered or produced by the hand of man and is in a recombinant bacterial or plant host cell. For example, a recombinant polynucleotide may be a polynucleotide isolated from a genome, a cDNA produced by the reverse transcription of an RNA, a synthetic nucleic acid molecule or an artificial combination of two otherwise separated segments of sequence, e.g., by chemical synthesis or by the manipulation of isolated segments of polynucleotides by genetic engineering techniques.

The term “Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

Optimal alignment of sequences for comparison may be conducted by computerized implementations of algorithms known in the art (GAP, BESTFIT, BLAST, PASTA, and TFASTA in the Wisconsin Genetics Software Package, Genetics Computer Group (GCG), 575 Science Dr., Madison, Wis.), or by inspection.

“Percentage of sequence identity,” as used herein, is determined by comparing two optimally aligned sequences over a comparison window, where the fragment of the polynucleotide or amino acid sequence in the comparison window may comprise additions or deletions (e.g., gaps or overhangs) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

Optimal alignment of sequences for comparison may be conducted by computerized implementations of algorithms known in the art (GAP, BESTFIT, BLAST, PASTA, and TFASTA in the Wisconsin Genetics Software Package, Genetics Computer Group (GCG), 575 Science Dr., Madison, Wis.), or by inspection.

The term “substantial sequence identity” between nucleotide sequences used herein refers to polynucleotide comprising a sequence that has at least 65% sequence identity, preferably at least 69% to 77% sequence identity compared to the reference sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “Operably linked” means any linkage, irrespective of orientation or distance, between a regulatory sequence and coding sequence, where the linkage permits the regulatory sequence to control expression of the coding sequence. The term “operably linked” further means that the nucleic acid sequences being linked are contiguous and, where necessary to join two protein coding regions, contiguous and in the same reading frame. The term “operably linked” also refers to a functional linkage between a promoter and a second sequence, wherein the promoter sequence initiates and mediates transcription of the DNA sequence corresponding to the second sequence.

As used herein, “heterologous DNA coding sequence” or “heterologous nucleic acid” or “heterologous polynucleotide” means any coding sequence other than the one that naturally encodes the Cry2Ai protein, or any homolog of the Cry2Ai protein.

As used herein, “coding region” refers to that portion of a gene, a DNA or a nucleotide sequence which codes for a protein. The term “non-coding region” refers to that portion of a gene a DNA or a nucleotide sequence that is not a coding region.

As used herein, the terms “encoding” or “encoded” when used in the context of a specified nucleic acid mean that the nucleic acid comprises the requisite information to direct translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The terms “polypeptide,” “peptide,” and “protein” are used interchangeably herein to refer to a polymer composed of amino acid residues related naturally occurring structural variants, and synthetic non-naturally occurring analogs thereof linked via peptide bonds. Synthetic polypeptides can be synthesized, for example, using an automated polypeptide synthesizer. The terms apply to amino acid polymers in which one or more amino acid residues is an artificial chemical analogue of a corresponding naturally occurring amino acid, as well as to naturally occurring amino acid polymers. The terms “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass known analogues of natural amino acids that can function in a similar manner as naturally occurring amino acids.

The term “protein” typically refers to large polypeptides. The term “peptide” typically refers to short polypeptides. However, the term “polypeptide” is used herein to refer to any amino acid polymer comprised of two or more amino acid residues linked via peptide bonds.

As used herein, “expression cassette” means a genetic module comprising a gene and the regulatory regions necessary for its expression, which may be incorporated into a vector.

A “vector” is a composition of matter which comprises nucleic acid molecule and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

The term “Expression vector” refers to a vector comprising a recombinant nucleic acid comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant nucleic acid.

The term “host cell” as used herein refers to a cell which contains a vector and supports the replication and/or expression of the expression vector is intended. Host cells may be prokaryotic cells such as E. coli, or eukaryotic cells such as yeast, insect, amphibian, or mammalian cells, or monocotyledonous or dicotyledonous plant cells. An example of a monocotyledonous host cell is a rice host cell and an example of a dicotyledonous host cell is eggplant or tomato host cell. When possible, the sequence is modified to avoid predicted hairpin secondary mRNA structures.

The term “toxin” as used herein refers to a polypeptide showing pesticidal activity or insecticidal activity. “Bt” or “Bacillus thuringiensis” toxin is intended to include the broader class of Cry toxins found in various strains of Bt, which includes such toxins.

The term “probe” or “sample probe” refers to a molecule that is recognized by its complement or a particular microarray element. Examples of probes that can be investigated by this invention include, but are not limited to, DNA, RNA, oligonucleotides, oligosaccharides, polysaccharides, sugars, proteins, peptides, monoclonal antibodies, toxins, viral epitopes, hormones, hormone receptors, enzymes, enzyme substrates, cofactors, and drugs including agonists and antagonists for cell surface receptors.

As used herein, the term “complementary” or “complement” refer to the pairing of bases, purines and pyrimidines that associate through hydrogen bonding in double stranded nucleic acid. The following base pairs are complementary: guanine and cytosine; adenine and thymidine; and adenine and uracil. The terms as used herein include complete and partial complementarity.

As used herein, the term “hybridization” refers to a process in which a strand of nucleic acid joins with a complementary strand through base pairing. The conditions employed in the hybridization of two non-identical, but very similar, complementary nucleic acids vary with the degree of complementarity of the two strands and the length of the strands. Thus the term contemplates partial as well as complete hybridization. Such techniques and conditions are well known to practitioners in this field.

The terms “insecticidal activity” and “pesticidal activity” are used interchangeably herein to refer to activity of an organism or a substance (such as, for example, a protein) that can be measured by, but is not limited to, pest mortality, pest weight loss, pest repellency, and other behavioural and physical changes of a pest after feeding and exposure for an appropriate length of time. Thus, an organism or substance having pesticidal activity adversely impacts at least one measurable parameter of pest fitness. For example, “insecticidal proteins” are proteins that display insecticidal activity by themselves or in combination with other proteins.

As used herein, the term “affecting insect pests” refers to controlling changes in insect feeding, growth, and/or behaviour at any stage of development, including but not limited to killing the insect, retarding growth, preventing reproductive capability, antifeedant activity, and the like.

As used herein, the term “pesticidally effective amount” connotes a quantity of a substance or organism that has pesticidal activity when present in the environment of a pest. For each substance or organism, the pesticidally effective amount is determined empirically for each pest affected in a specific environment. Similarly, an “insecticidally effective amount” may be used to refer to a “pesticidally effective amount” when the pest is an insect pest.

As used herein, the terms “transformed plant” and “transgenic plant” refer to a plant that comprises one or more heterologous polynucleotide within its genome. The heterologous polynucleotide(s) is stably integrated within the genome of a transgenic or transformed plant such that the polynucleotide is passed on to successive generations. The heterologous polynucleotide may be integrated into the genome alone or as part of a recombinant DNA molecule.

It is to be understood that as used herein the term “transgenic” includes any plant cell, plant cell line, callus, tissue, a plant part, or a plant the genotype of which has been altered by the presence of one or more heterologous nucleic acid. The term includes those transgenics initially obtained using genetic transformation method known in the art as well as those created by sexual crosses or asexual propagation from the initial transgenic.

The term “initial transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

As used herein, the term “plant” includes whole plants, plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like and progeny thereof. Parts of transgenic plants are within the scope of the embodiments and comprise, for example, plant cells, protoplasts, tissues, callus, embryos as well as flowers, stems, fruits, leaves, and roots originating in transgenic plants or their progeny previously transformed with a DNA molecule of the embodiments and therefore consisting at least in part of transgenic cells.

Bacillus thuringiensis Cry Gene and Codon Optimization

Approximately about 400 cry genes encoding δ-endotoxins have now been sequenced (Crickmore, N. 2005. Using worms to better understand how Bacillus thuringiensis kills insects. Trends in Microbiology, 13(8): 347-350). The various δ-endotoxins have been classified into classes (Cry 1, 2, 3, 4, etc.) on the basis of amino acid sequence similarities. These classes are composed of several subclasses (Cry1A, Cry1B, Cry1C, etc.), which are themselves subdivided into subfamilies or variants (Cry1Aa, Cry1Ab, Cry1Ac, etc.). The genes of each class are more than 45% identical to each other. The product of each individual cry gene generally has a restricted spectrum of activity, limited to the larval stages of a small number of species. However, it has not been possible to establish a correlation between the degree of identity of Cry proteins and their spectrum of activity. The Cry1Aa and Cry1Ac proteins are 84% identical, but only Cry1Aa is toxic to Bombyx mori (L.). Conversely, Cry3Aa and Cry7Aa, which are only 33% identical, are both active against the Colorado potato beetle, Leptinotarsa decemlineata. Other Cry toxins are not active against insects at all, but are active against other invertebrates. For example, the Cry5 and Cry6 protein classes are active against nematodes. More recently, binary toxins from Bt designated as Cry34Ab1/Cry35Ab1, active against various Coleopteran insect pests of the Chrysomelidae family have also been characterized. They have been assigned a Cry designation, although they have little homology to the other members of the Cry toxin family.

To achieve desired expression levels of heterologous proteins in transgenic plants it has been found beneficial to alter the native, sometimes referred to as wild-type or original genomic DNA coding sequence in various ways, so that codon usage more closely matches the codon usage of the host plant species, similarly the G+C content of the coding sequence more closely matches the G+C content of the host plant species.

One skilled in the art of plant molecular biology will understand that multiple DNA sequences may be designed to encode a single amino acid sequence. A common means of increasing the expression of a coding region for a protein of interest is to modify the coding region in such a manner that its codon composition resembles the overall codon composition of the host in which the gene is targeted to be expressed.

A genomic/native nucleic acid may be optimized for increased expression in the host organism. Thus, where the host organism is a plant, the synthetic nucleic acids can be synthesized using plant-preferred codons for improved expression. For example, although nucleic acid sequences of the embodiments may be expressed in both monocotyledonous and dicotyledonous plant species, sequences can be modified to account for the specific codon preferences and GC content preferences of monocotyledons or dicotyledons as these preferences have been shown to differ (Murray et al. (1989) Nucleic Acids Res. 17:477-498). Thus, the rice preferred codon for a particular amino acid may be derived from known gene sequences from rice and the eggplant preferred codon for a particular amino acid may be derived from known gene sequences from eggplant.

Additional sequence modifications are known to enhance gene expression in a cellular host. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given cellular host, as calculated by reference to known genes expressed in the host cell.

Vaeck et al., (Vaeck M, Reynaerts A, Höfte H, Jansens S, De Beukeleer M, Dean C, Zabeau M, Van Montagu M, Leemans J (1987) Transgenic plants protected from insect attack. Nature 327:33-37) reported production of insect resistant transgenic tobacco plants expressing Bt cry1Ab gene for protection against the European corn borer, one of the main pest attacking maize in the US and Europe. However, despite the use of strong promoters, toxin production in the plants was initially too weak for effective agricultural use (Koziel G M, Beland G L, Bowman C, Carozzi N B, Crenshaw R, Crossland L, Dawson J, Desai N, Hill M, Kadwell S, Launis K, Maddox D, McPherson K, Heghji M, Merlin E, Rhodes R, Warren G, Wright M, Evola S (1993) Field performance of elite transgenic maize plants expressing an insecticidal protein derived from Bacillus thuringiensis. Biotechnology 11:194-200). Unlike plant genes, Bt genes have a high A+T content (66%), which is a suboptimal codon usage for plants, and potentially leads to missplicing or premature termination of transcription (De la Riva and Adang, 1996).

Perlak et al., (Perlak F J, Fuchs R L, Dean D A, McPherson S L, Fishhoff D A (1991) Modification of the coding sequences enhances plant expression of insect control protein genes, Proc. Natl. Acad. Sci. (USA) 88:3324-3328.) modified the coding sequence of cry genes without modifying the encoded peptide sequence to ensure optimal codon usage for plants that allowed two fold toxin productions in plants compared to the native gene. This strategy has been successfully used in many plants such as cotton, rice and maize transformed with modified cry1 genes and potato transformed with a modified cry3A gene. Bt maize and Bt cotton are cultivated on a large scale, throughout the world.

Thus, naturally existing codon bias among organisms leads to sub-optimal expression of genes in heterologous organism. In the present invention native cry2Ai gene from Bacillus thuringiensis was reconfigured in-silico for optimum expression of recombinant protein in plants including dicotyledonous and monocotyledons plants. While designing by multivariate analysis the rare and highly rare codons were substituted with the highly preferable codons of dicot/monocot plants. The reconfigured synthetic gene designed by gene designer tool was manually checked for the rare codon usage, stability of secondary structure of mRNA, any start of secondary transcription of gene, to avoid expression of truncated proteins. The structure and stability of optimized mRNA was checked and confirmed by mRNA optimizer.

Particular aspect of the invention provides codon-optimised synthetic nucleic acid encoding Cry2Ai insecticidal proteins, insecticidal compositions, polynucleotide constructs, recombinant nucleotide sequence, recombinant vector, transformed microorganisms and plants comprising the nucleic acid molecule of the invention. These compositions find use in methods for controlling insect pests, especially crop plant insect pests.

The codon optimized synthetic nucleotide sequence disclosed herein can be fused with a variety of promoters, including constitutive, inducible, temporally regulated, developmentally regulated, tissue-preferred and tissue-specific promoters to prepare recombinant DNA molecules. The codon optimized synthetic cry2Ai nucleotide sequences disclosed herein (coding sequence) provides substantially higher levels of expression in a transformed plant, when compared with a native cry2Ai gene. Accordingly, plants resistant to Lepidopteran pests, such as Helicoverpa armigera—the cotton bollworm and corn earworm, Cnaphalocrocis medinalis—the rice leaffolder, and Scirpophaga incertulas—the rice yellow stem borer and Pectinophora gossypiella can be produced.

One embodiment of the present invention provides codon optimized synthetic cry2Ai nucleotide sequences with plant preferred codons. Another embodiment of the present invention provides expression of the codon optimized synthetic cry2Ai nucleotide sequence(s) in plants such as cotton, eggplant, rice, tomato and maize. Another embodiment of the present invention provides DNA expression cassettes, plant transformation vectors comprising the synthetic cry2Ai nucleotide sequence(s) of the invention. Another embodiment of the present invention provides compositions comprising the codon optimized synthetic cry2Ai nucleotide sequence(s) disclosed herein or the insecticidal polypeptide encoded by the codon optimized synthetic cry2Ai nucleotide sequence(s) disclosed herein. The composition disclosed herein may be pesticidal and or insecticidal compositions comprising the pesticidal and/or insecticidal proteins/polypeptide of the invention. Another embodiment provides transgenic plants comprising the codon optimized synthetic cry2Ai nucleotide sequence(s) of the invention expressing Cry2Ai toxin protein.

In particular, the present invention provides the codon optimized synthetic nucleotide sequence as set forth in SEQ ID NO: 2, SEQ ID NO: 3, SEQ ID NO: 4, SEQ ID NO: 5, and SEQ ID NO: 6, wherein the said nucleotide encodes the insecticidal Cry2Ai protein having amino acid sequence as set forth in SEQ ID NO: 1.

In some embodiment the invention further provides plants and microorganisms transformed with the codon optimized synthetic cry2Ai nucleotide sequence(s) disclosed herein, and methods involving the use of such nucleotide sequence(s), pesticidal compositions, transformed organisms, and products thereof in affecting insect pests.

The polynucleotide sequences of the embodiments may be used to transform any organism for example plants and microorganism such as Bacillus thuringiensis to produce the encoded insecticidal and/or pesticidal proteins. Methods are provided that involve the use of such transformed organisms to affect or control plant insect pests. The nucleic acids and nucleotide sequences of the embodiments may also be used to transform organelles such as chloroplasts. The method of transformation of the desired organism is well known in the art that enable a person skilled in the art to perform the transformation using the nucleotide sequences disclosed in the present invention.

The nucleotide sequences of the embodiments encompass nucleic acid or nucleotide sequences that have been optimized for expression by the cells of a particular organism, for example nucleic acid sequences that have been back-translated (i.e., reverse translated) using plant-preferred codons based on the amino acid sequence of a polypeptide having pesticidal activity.

The disclosure provides codon optimised synthetic nucleic acid/nucleotide sequences encoding insecticidal Cry2Ai proteins. The synthetic coding sequences are particularly adapted for use in expressing the proteins in Dicotyledonous (dicot) and monocotyledons (monocot) plants such as rice, tomato, eggplant, maize, cotton, and legumes.

The disclosure provides synthetic nucleotide sequences encoding Cry2Ai protein that are particularly adapted to express well in plants. The disclosed codon optimised synthetic nucleotide sequences utilize plant-optimized codons roughly in the same frequency at which they are utilized, on average, in genes naturally occurring in the plant species. The disclosure further includes codon optimised synthetic nucleotide sequence for conferring insect resistance in plants.

For plant transformation selectable marker genes are used in the present disclosure. DNA construct and transgenic plants containing the synthetic sequences disclosed herein are taught as are methods and compositions for using the agriculturally important plants.

The protein encoded by the codon optimised synthetic nucleotide sequences each exhibit lepidopteron species inhibitory biological activity. Dicotyledonous and/or monocotyledons plants can be transformed with each of the nucleotide sequences disclosed herein alone or in combinations with other nucleotide sequences encoding insecticidal agents such as proteins, crystal proteins, toxins, and/or pest specific double stranded RNA's designed to suppress genes within one or more target pests, and the like to achieve a means of insect resistance management in the field that has not feasible before by merely using the known lepidopteran insecticidal proteins derived from Bacillus thuringiensis strains.

The codon optimised synthetic nucleotide sequences of the present invention can also be used in plants in combination with other types of nucleotide sequences encoding insecticidal toxins for achieving plants transformed to contain at least one means for controlling one or more of each of the common plant pests selected from the groups consisting of lepidopteran insect pests, coleopteran insect pests, piercing and sucking insect pests, and the like.

### Regulatory Sequences

Transcriptional and translational regulatory signals include, but are not limited to, promoters, transcriptional initiation start sites, operators, activators, enhancers, other regulatory elements, ribosomal binding sites, an initiation codon, termination signals, and the like.

The polynucleotide/DNA construct will include in the 5′ to 3′ direction of transcription: a transcriptional and translational initiation region (i.e., a promoter), a DNA sequence of the embodiments, and a transcriptional and translational termination region (i.e., termination region) functional in the organism serving as a host. The transcriptional initiation region (i.e., the promoter) may be native, analogous, foreign or heterologous to the host organism and/or to the sequence of the embodiments. Additionally, the promoter may be the natural sequence or alternatively a synthetic sequence. The term “foreign” as used herein indicates that the promoter is not found in the native organism into which the promoter is introduced. Where the promoter is “foreign” or “heterologous” to the sequence of the embodiments, it is intended that the promoter is not the native or naturally occurring promoter for the operably linked sequence of the embodiments.

A number of promoters can be used in the practice of the embodiments. The promoters can be selected based on the desired outcome. The codon optimized nucleotide sequence of the invention can be combined with constitutive, tissue-preferred, inducible, or other promoters for expression in the host organism. Suitable constitutive promoters for use in a plant host cell include, for example, the core CaMV 35S promoter; rice actin; ubiquitin; ALS promoter etc.

Depending on the desired outcome, it may be beneficial to express the gene from an inducible promoter. Of particular interest for regulating the expression of the nucleotide sequences of the embodiments in plants are wound-inducible promoters. Such wound-inducible promoters, may respond to damage caused by insect feeding, and include potato proteinase inhibitor (pin II) gene; wun1 and wun2, win1 and win2; WIP1; MPI gene etc.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, β-1,3-glucanase, chitinase, etc.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters.

A promoter that has “preferred” expression in a particular tissue is expressed in that tissue to a greater degree than in at least one other plant tissue. Some tissue-preferred promoters show expression almost exclusively in the particular tissue. Tissue-preferred promoters can be utilized to target enhanced pesticidal protein expression within a particular plant tissue. Such promoters can be modified, if necessary, for weak expression.

Example of some of the tissue specific promoters includes but is not limited to leaf-preferred promoters, root specific or root preferred promoters, seed specific or seed preferred promoters, pollen specific promoters, and pith specific promoters.

Root-preferred or root-specific promoters are known and can be selected from the available from the literature or isolated de novo from various compatible species.

“Seed-preferred” promoters include both “seed-specific” promoters (those promoters active during seed development such as promoters of seed storage proteins) as well as “seed-germinating” promoters (those promoters active during seed germination). Gamma-zein and Glob-1 are endosperm-specific promoters. For dicots, seed-specific promoters include, but are not limited to β.-phaseolin, β.-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc.

Where low level expression is desired, weak promoters will be used. Generally, the term “weak promoter” as used herein refers to a promoter that drives expression of a coding sequence at a low level. Such weak constitutive promoters include, for example the core promoter of the Rsyn7 promoter, the core 35S CaMV promoter etc.

Termination regions are available from the Ti-plasmid of A. tumefaciens, such as the octopine synthase (OCS) and nopaline synthase (NOS) termination regions.

The DNA expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region); potyvirus leaders, for example, TEV leader (Tobacco Etch Virus), MDMV leader (Maize Dwarf Mosaic Virus), untranslated leader from the coat protein mRNA of alfalfa mosaic virus (AMV RNA 4); tobacco mosaic virus leader (TMV); and maize chlorotic mottle virus leader (MCMV).

In one specific embodiment of the invention disclosed and claimed herein, the tissue-preferred or tissue-specific promoter is operably linked to a synthetic DNA sequence of the disclosure encoding the insecticidal protein, and a transgenic plant stably transformed with at least one such recombinant molecule. The resultant plant will be resistant to particular insects which feed on those parts of the plant in which the DNA(s) is (are) expressed.

### Selectable Marker Gene

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (nptII) and hygromycin phosphotransferase (hptII), as well as genes conferring resistance to herbicidal compounds, such as glufosinate ammonium, bromoxynil, imidazolinones, and 2,4-dichlorophenoxyacetate (2,4-D). Additional examples of suitable selectable marker genes include, but are not limited to, genes encoding resistance to chloramphenicol, methotrexate, streptomycin, spectinomycin, bleomycin, sulphonamide, bromoxynil, glyphosate, phosphinothricin.

The above list of selectable marker genes is not meant to be limiting. Any selectable marker gene can be used in the embodiments.

### DNA Constructs and Vectors

The codon optimized synthetic nucleotide sequences of the inventions are provided in DNA constructs for expression in the organism of interest. The construct includes 5′ and 3′ regulatory sequences operably linked to a sequence of the invention.

Such a polynucleotide construct is provided with a plurality of restriction sites for insertion of the DNA sequences encoding Cry2Ai toxin protein sequence to be under the transcriptional regulation of the regulatory regions. The polynucleotide construct may additionally contain selectable marker genes. The construct may additionally contain at least one additional gene to be co-transformed into the desired organism. Alternatively, the additional gene(s) can be provided on multiple polynucleotide constructs.

In preparing the DNA construct/expression cassette, the various DNA fragments may be manipulated so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

According to the present invention, the DNA construct/expression cassette disclosed herein may be inserted to the recombinant expression vector. The expression “recombinant expression vector” means a bacteria plasmid, a phage, a yeast plasmid, a plant cell virus, a mammalian cell virus, or other vector. In general, as long as it can be replicated and stabilized in a host, any plasmid or vector can be used. Important characteristic of the expression vector is that it has a replication origin, a promoter, a marker gene, and a translation control element.

A large number of cloning vectors comprising a replication system in E. coli and a marker that permits selection of the transformed cells are available for preparation for the insertion of foreign genes into higher plants. The vectors comprise, for example, pBR322, pUC series, M13mp series, pACYC184, inter alia. Accordingly, the DNA fragment having the sequence encoding the Bt toxin protein can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted.

The expression vector comprising the codon optimized nucleotide sequence of the disclosure and a suitable signal for regulating transcription/translation can be constructed by a method which is well known to a person in the art. Examples of such method include an in vitro recombination DNA technique, a DNA synthesis technique, and an in vivo recombination technique. The DNA sequence can be effectively linked to a suitable promoter in the expression vector in order to induce synthesis of mRNA. Furthermore, the expression vector may contain, as a site for translation initiation, a ribosome binding site and a transcription terminator.

A preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector are currently used for transferring a hybrid gene to protoplasts that can produce a new plant by appropriately inserting a plant cell or hybrid DNA to a genome of a plant.

Expression vector may comprise at least one selectable marker gene. The selectable marker gene is a nucleotide sequence having a property based on that it can be selected by a common chemical method. Every gene which can be used for the differentiation of transformed cells from non-transformed cell can be a selective marker. Example includes, a gene resistant to herbicide such as glyphosate and phosphintricin, and a gene resistant to antibiotics such as kanamycin, hygromycin, G418, bleomycin, and chloramphenicol, but not limited thereto.

For the recombinant vector of the present invention, a promoter can be any of CaMV 35S, actin, or ubiquitin promoter, but not limited thereto. Since a transformant can be selected with various mechanisms at various stages, a constitutive promoter can be preferable for the present invention. Therefore, a possibility for choosing a constitutive promoter is not limited herein.

For the recombinant vector of the present invention, any conventional terminator can be used. Examples thereof include nopaline synthase (NOS), rice α-amylase RAmy1 A terminator, phaseoline terminator, and a terminator for optopine gene of Agrobacterium tumefaciens, etc., but are not limited thereto. Regarding the necessity of terminator, it is generally known that such region can increase reliability and an efficiency of transcription in plant cells. Therefore, the use of terminator is highly preferable in view of the contexts of the present invention.

One skilled in the art will know that the DNA construct and vector disclosed herein can be used for production of insect resistant transgenic plants and/or production of insecticidal composition, wherein the composition comprises may comprise Bacillus thuringiensis cells comprising the said nucleotide sequence or any other microorganism capable of expressing the nucleotide sequence disclosed herein to produce the Cry2Ai insecticidal protein.

### Recombinant Cell

The embodiments further encompass a microorganism that is transformed with at least one codon optimized nucleic acid of the invention, with an expression cassette comprising the nucleic acid, or with a vector comprising the expression cassette. In some embodiments, the microorganism is one that multiplies on plants. An embodiment of the invention relates to an encapsulated pesticidal protein which comprises a transformed microorganism capable of expressing the Cry2Ai protein of the invention.

A further embodiment relates to a transformed organism such as an organism selected from the group consisting of plant and insect cells, bacteria, yeast, baculoviruses, protozoa, nematodes, and algae. The transformed organism comprises a codon optimized synthetic

DNA molecule of the invention, an expression cassette comprising the said DNA molecule, or a vector comprising the said expression cassette, which may be stably incorporated into the genome of the transformed organism.

It is recognized that the genes encoding the Cry2Ai protein can be used to transform insect pathogenic organisms. Such organisms include baculoviruses, fungi, protozoa, bacteria, and nematodes.

The codon optimized synthetic nucleotide sequence(s) encoding the Cry2Ai protein of the embodiments may be introduced via a suitable vector into a microbial host, and said host applied to the environment, or to plants or animals. The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid, or mitochondrial DNA), converted into an autonomous replicon, or transiently expressed (e.g., transfected mRNA).

A number of ways are available for introducing a foreign DNA expressing the pesticidal protein into the microorganism host under conditions that allow for stable maintenance and expression of the DNA. For example, expression cassettes can be constructed which include the nucleotide constructs of interest operably linked with the transcriptional and translational regulatory signals for expression of the nucleotide constructs, and a nucleotide sequence homologous with a sequence in the host organism, whereby integration will occur, and/or a replication system that is functional in the host, whereby integration or stable maintenance will occur.

### Plant Transformation Methods and Production of Transgenic Plants

The codon optimized synthetic nucleotide sequence (DNA sequence) of the present invention encoding Bt Cry2Ai toxin protein can be inserted into plant cells using a variety of techniques which are well known in the art. Once the inserted DNA has been integrated in the plant genome, it is relatively stable. The transformation vector normally contains a selectable marker that confers on the transformed plant cells resistance to a biocide or an antibiotic, such as Kanamycin, Bialaphos, G418, Bleomycin, or Hygromycin. The individually employed marker should accordingly permit the selection of transformed cells rather than cells that do not contain the inserted DNA.

A large number of techniques are available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA.

Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the Right and Left T-DNA border regions. They can be transformed directly into Agrobacteria. The Agrobacterium used as host cell is to comprise a plasmid carrying a virulence (vir) region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, leaf pieces, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The cells that have been transformed may be grown into plants in accordance with conventional ways. These plants may then be grown, and either pollinated with the same transformed strain or different strains, and the resulting hybrid having constitutive or inducible expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

The plant transformation methods of the present invention involve introducing the polynucleotide(s) of the invention into a plant and do not depend on a particular method for introducing a polynucleotide(s) into a plant. Methods for introducing polynucleotide(s) into plants are known in the art including, but not limited to, stable transformation methods, transient transformation methods, and virus-mediated methods.

In one embodiment of the present invention, plants were transformed with the codon optimized synthetic nucleotide sequence(s) disclosed herein. Some non-limiting example of transformed plants is fertile transgenic rice and tomato plant comprising the codon optimized synthetic nucleotide sequence(s) of the invention encoding a Cry2Ai protein. Method of transformation of rice, and tomato are known in the art. Various other plants can also be transformed using the codon optimized synthetic nucleotide sequence(s) disclosed herein.

“Stable transformation” is intended to mean that the nucleotide construct introduced into a plant integrates into the genome of the plant and is capable of being inherited by the progeny thereof. “Transient transformation” is intended to mean that a polynucleotide is introduced into the plant and does not integrate into the genome of the plant or a polypeptide is introduced into a plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection, electroporation, Agrobacterium-mediated transformation and ballistic particle acceleration.

In one embodiment of the invention, the codon optimized synthetic nucleotide sequence(s) of the invention encoding the Cry2Ai toxin is expressed in a higher organism, e.g., a plant using the codon optimized nucleotide sequence of the disclosure. In this case, transgenic plants expressing effective amounts of the toxin protect themselves from insect pests. When the insect starts feeding on such a transgenic plant, it also ingests the expressed toxin. This will deter the insect from further biting into the plant tissue or may even harm or kill the insect. The nucleotide sequence of the invention is inserted into an expression cassette, which is then stably integrated in the genome of the plant.

The embodiments also encompass transformed or transgenic plants comprising at least one nucleotide sequence of the embodiments. In some embodiments, the plant is stably transformed with a nucleotide construct comprising at least one nucleotide sequence of the embodiments operably linked to a promoter that drives expression in a plant cell.

While the embodiments do not depend on a particular biological mechanism for increasing the resistance of a plant to a plant pest, expression of the nucleotide sequences of the embodiments in a plant can result in the production of the pesticidal proteins of the embodiments and in an increase in the resistance of the plant to a plant pest. The plants of the embodiments find use in agriculture methods for affecting insect pests. Certain embodiments provide transformed crop plants, such as, for example rice and tomato plants, which find use in methods for affecting insect pests of the plant, such as, for example, various lepidopteran insects, including Cnaphalocrocis medinalis—the rice leaffolder, and Scirpophaga incertulas—the rice yellow stem borer.

A “subject plant or plant cell” is one in which genetic alteration, such as transformation, has been affected as to a gene of interest, or is a plant or plant cell which is descended from a plant or cell so altered and which comprises the alteration. A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

Transfer (or introgression) of the cry2Ai nucleotides disclosed herein determined trait into inbred plants such as cotton, rice, eggplant (brinjal), tomato and legume lines can be achieved by recurrent selection breeding, for example by backcrossing. In this case, a desired recurrent parent is first crossed to a donor inbred (the non-recurrent parent) that carries the nucleotide sequence disclosed herein for the cry2Ai determined traits. The progeny of this cross is then backcrossed with the recurrent parent followed by selection in the resultant progeny for the desired trait(s) to be transferred from the non-recurrent parent. After three, preferably four, more preferably five or more generations of backcrosses with the recurrent parent with selection for the desired trait(s), the progeny will be heterozygous for loci controlling the trait(s) being transferred, but will be like the recurrent parent for most or almost all other genes.

The embodiments further relate to plant-propagating material of a transformed plant of the embodiments including, but not limited to, seeds, tubers, corms, bulbs, leaves, and cuttings of roots and shoots.

The class of plants that can be used in the methods of the embodiments is generally as broad as the class of higher plants amenable to transformation techniques, including but not limited to monocotyledonous and dicotyledonous plants. Examples of plants of interest include, but are not limited to grains, cereal, vegetable, oil, fruits, ornamentals, Turfgrasses and others. For example, rice (Oryza sativa, Oryza spp.), corn (Zea mays), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum, Triticum sp.), oats (Avena sativa), barley (Hordeum vulgare L), sugarcane (Saccharum spp.), cotton (Gossypium hirsutum, Gossypium barbadense, Gossypium sp.), tomatoes (Lycopersicon esculentum), brinjal/eggplant (Solanum melongena), potato (Solanum tuberosum), sugar beets (Beta vulgaris), sweet potato (Ipomoea batatus), cassava (Manihot esculenta), lettuce (Lactuca sativa), cabbage (Brassica oleracea var. capitata), cauliflower (Brassica oleracea var. botrytis), broccoli (Brassica oleracea var. indica), Brassica sp. (e.g., B. napus, B. rapa, B. juncea), particularly those Brassica species useful as sources of seed oil, soybean (Glycine max), sunflower (Helianthus annuus), safflower (Carthamus tinctorius), peanuts (Arachis hypogaea), pigeon-pea (Cajanus cajan), Chickpea (Cicer arietinum), green beans (Phaseolus vulgaris), lima beans (Phaseolus limensis), pea (Pisum sativum), peas (Lathyrus spp.), cucumber (Cucumis sativus), cantaloupe (Cucumis cantalupensis), and musk melon (Cucumis melo), alfalfa (Medicago sativa), tobacco (Nicotiana tabacum) and Arabidopsis (Arabidopsis thaliana).

Plants of interest include grain plants that provide seeds of interest, oil-seed plants, and leguminous plants. Seeds of interest include grain seeds, such as corn, wheat, barley, rice, sorghum, rye, millet, etc. Oil-seed plants include cotton, soybean, safflower, sunflower, Brassica, maize, alfalfa, palm, coconut, flax, castor, olive etc. Leguminous plants include beans and peas. Beans include guar, locust bean, fenugreek, soybean, garden beans, cowpea, mungbean, lima bean, fava bean, lentils, chickpea, etc.

In certain embodiments at least one codon optimized synthetic nucleotide sequence(s) of the invention can be stacked with any combination of polynucleotide sequences of interest in order to create plants with a desired phenotype. For example, the codon optimized synthetic nucleotide sequence may be stacked with any other polynucleotide encoding polypeptide having pesticidal and/or insecticidal activity, such as other Bt toxic proteins and the like. The combinations generated can also include multiple copies of any one of the polynucleotide of interest. The nucleotide sequences of the embodiments can also be stacked with any other gene or combination of genes to produce plants with a variety of desired trait combinations including but not limited to traits desirable for animal feed such as high oil genes, balanced amino acids, abiotic stress resistance etc.

The nucleotide sequences of the invention can also be stacked with traits desirable for disease or herbicide resistance, avirulence and disease resistance genes, acetolactate synthase (ALS), inhibitors of glutamine synthase such as phosphinothricin or basta (e.g., bar gene), glyphosate resistance, traits desirable for processing or process products such as high oil, modified oils, modified starches (e.g., ADPG pyrophosphorylases (AGPase), starch synthases (SS), starch branching enzymes (SBE) and starch debranching enzymes (SDBE)). One could also combine the polynucleotides of the embodiments with polynucleotides providing agronomic traits such as male sterility, stalk strength, flowering time, or transformation technology traits such as cell cycle regulation or gene targeting.

These stacked combinations can be created by any method including but not limited to cross breeding plants by any conventional, genetic transformation or any other method known in the art. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of the polynucleotide of interest. This may be combined with any combination of other suppression cassettes or over-expression cassettes to generate the desired combination of traits in the plant. It is further recognized that polynucleotide sequences can be stacked at a desired genomic location using a site-specific recombination system.

### Analysis of Transgenic Plants

### Polymerase Chain Reaction (PCR)

The present invention provides a method for PCR amplification of a fragment of the codon optimized nucleotide sequence as set forth in SEQ ID NO: 2 disclosed in the invention encoding the Cry2Ai protein having amino acid sequence as set forth in SEQ ID NO: 1, comprising amplifying DNA by PCR in presence of the primer set as forth in SEQ ID NO: 7-8. Similarly a fragment of the codon optimized nucleotide sequence as set forth in SEQ ID NO: 3 to 6 disclosed in the invention can be amplified using the primers specific to the said nucleotide sequence. A person skilled in the art can design the primer set for amplification of the said nucleotides. Protocols and conditions for the PCR amplification of a DNA fragment from template DNA are described elsewhere herein or are otherwise known in the art.

Oligonucleotide primers can be designed for use in PCR reactions to amplify corresponding DNA sequences from codon optimised DNA sequences of the invention. Methods for designing PCR primers and PCR cloning are generally known in the art and are disclosed in Sambrook et al. (1989) Molecular Cloning: A Laboratory Manual (2nd ed., Cold Spring Harbor Laboratory Press, Plainview, N.Y.), hereinafter “Sambrook”. Known methods of PCR include, but are not limited to, methods using paired primers, nested primers, single specific primers, degenerate primers, gene-specific primers, vector-specific primers, partially-mismatched primers, and the like.

In one embodiment, the present invention provides a primer set suitable for the PCR amplification of a fragment of the codon optimised synthetic nucleotide sequence(s) of the present invention that encode a pesticidal polypeptide and methods of using the primer set in the PCR amplification of the DNA. The primer sets comprise forward and reverse primers that have been designed to anneal to the nucleotide sequences of the present invention.

The primer sets for amplification of the nucleotide sequence as set forth in SEQ ID NO: 2 of the invention comprise forward and reverse primers as set forth in SEQ ID NO: 7 and SEQ ID NO: 8.

### Southern Hybridization

In hybridization techniques, all or part of a known nucleotide sequence is used as a probe that selectively hybridizes to other corresponding nucleotide sequences present in a population of cloned genomic DNA fragments or from a chosen organism. The hybridization probes may be PCR amplified DNA fragments of the codon optimised DNA sequence(s) of the present invention, or linerarized plasmid containing the nucleotide sequence(s) or other oligonucleotides capable of hybridizing to the corresponding sequences of the synthetic nucleotide disclosed herein, and may be labelled with a detectable group such ³²P or any other detectable marker. Thus, for example, probes for hybridization can be made by labelling synthetic oligonucleotides based on the sequences of the embodiments. Methods for preparation of probes for hybridization generally known in the art and are disclosed in Sambrook.

For example, an entire sequence disclosed herein, or one or more portions thereof, may be used as a probe capable of specifically hybridizing to corresponding sequences. To achieve specific hybridization under a variety of conditions, such probes include sequences that are unique to the sequences of the embodiments and are generally at least about 10 or 20 nucleotides in length. Such probes may be used to amplify corresponding cry2Ai nucleotide sequence(s) of the nucleotide sequences by PCR.

Hybridization of such sequences may be carried out under stringent conditions. The term “stringent conditions” or “stringent hybridization conditions” as used herein refers to conditions under which a probe will hybridize to its target sequence to a detectably greater degree than to other sequences (e.g., at least 2-fold, 5-fold, or 10-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30% to 35% formamide, 1 M NaCl, 1% SDS (sodium dodecyl sulfate) at 37° C. and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a final wash in 0.1×SSC at 60° C. to 65° C. for at least about 20 minutes. Optionally, wash buffers may comprise about 0.1% to about 1% SDS. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tm (thermal melting point) can be approximated from the equation of Meinkoth and Wahl (1984) Anal. Biochem. 138:267-284: Tm=81.5° C.+16.6 (log M)+0.41 (% GC)-0.61 (% form)-500/L; where M is the molarity of monovalent cations, % GC is the percentage of guanosine and cytosine nucleotides in the DNA, “% form” is the percentage of formamide in the hybridization solution, and L is the length of the hybrid in base pairs. The T^{m} is the temperature (under defined ionic strength and pH) at which 50% of a complementary target sequence hybridizes to a perfectly matched probe. Washes are typically performed at least until equilibrium is reached and a low background level of hybridization is achieved, such as for 2 hours, 1 hour, or 30 minutes. T^{m} is reduced by about 1° C. for each 1% of mismatching; thus, I′ hybridization, and/or wash conditions can be adjusted to hybridize to sequences of the desired identity. For example, if sequences with 90% identity are sought, the T^{m} can be decreased 10° C. Generally, stringent conditions are selected to be about 5° C. lower than the T^{m} for the specific sequence and its complement at a defined ionic strength and pH. However, severely stringent conditions can utilize a hybridization and/or wash at 1, 2, 3, or 4° C. lower than the T^{m}; moderately stringent conditions can utilize a hybridization and/or wash at 6° C., 7° C., 8° C., 9° C., or 10° C. lower than the T^{m}; low stringency conditions can utilize a hybridization and/or wash at 11° C., 12° C., 13° C., 14° C., 15° C., or 20° C. lower than the T^{m}.

Using the equation, hybridization and wash compositions, and desired T^{m}, those of ordinary skill will understand that variations in the stringency of hybridization and/or wash solutions are inherently described. If the desired degree of mismatching results in a T^{m} of less than 45° C. (aqueous solution) or 32° C. (formamide solution), the SSC concentration can be increased so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See also Sambrook et al.

The present invention encompasses a nucleotide sequence complementary to the nucleotide sequence as set forth in SEQ ID NO: 2, or a nucleotide sequence which specifically hybridizes to at least 10 nucleotides of the nucleotide sequence as set forth in SEQ ID NO: 2 from nucleotide position 262 to 402 and/or 1471 to 1631 or a sequence complementary thereto. Those skilled in the art will know preparation of a probe based on the nucleotide sequences disclosed herein for nucleic acid hybridization.

### Protein Expression Assays

To determine the expression levels of protein of interest quantitatively various assays can be performed. The expression level of protein of interest may be measured directly, for example, by assaying for the level of the encoded protein in the plant. Methods for such assays are well-known in the art. For example, Northern blotting or quantitative reverse transcriptase-PCR (qRT-PCR) may be used to assess transcript levels, while western blotting, ELISA (enzyme-linked immunosorbent assay) assays, or enzyme assays may be used to assess protein levels.

In the present invention, Cry2Ai protein expression level in the transgenic plants comprising the codon optimised nucleotide sequence as disclosed in the invention was determined by using ELISA and it was surprisingly and unexpectedly found that the codon optimized synthetic DNA sequences as disclosed herein shows significant enhancement in Cry2Ai protein expression in the transgenic plants. The enhanced Cry2Ai protein expression in the transgenic plants thus obtained may be optimal for efficiently causing highest level of protection against the target insects. Thus, the codon optimized synthetic DNA sequences disclosed herein can be used for effective insect control in plants for enhanced resistance to insect pest thereby enhancing crop yield.

### Bioassay

A wide variety of bioassay techniques are known to one skilled in the art. General procedures include addition of the experimental compound or organism to the diet source in an enclosed container. Pesticidal activity can be measured by, but is not limited to, changes in mortality, weight loss, attraction, repellency and other behavioral and physical changes after feeding and exposure for an appropriate length of time. Bioassays described herein can be used with any feeding insect pest in the larval or adult stage.

### Insecticidal Composition

Biological pesticide is one of the most promising alternatives over conventional chemical pesticides, which offers less or no harm to the environments and biota. Bacillus thuringiensis (commonly known as Bt) is an insecticidal Gram-positive spore-forming bacterium producing crystalline proteins called delta-endotoxins (6-endotoxin) during its stationary phase or senescence of its growth. Bt was originally discovered from diseased silkworm (Bombyx mori) by Shigetane Ishiwatari in 1902. But it was formally characterized by Ernst Berliner from diseased flour moth caterpillars (Ephestia kuhniella) in 1915. The first record of its application to control insects was in Hungary at the end of 1920, and in Yugoslavia at the beginning of 1930s, it was applied to control the European corn borer. Bt, the leading biorational pesticide was initially characterized as an insect pathogen, and its insecticidal activity was ascribed largely or completely to the parasporal crystals. It is active against more than 150 species of insect pests. The toxicity of Bt culture lies in its ability to produce the crystalline protein, this observation led to the development of bioinsecticides based on Bt for the control of certain insect species among the orders Lepidoptera, Diptera, and Coleoptera.

Compositions of the embodiments find use in protecting plants, seeds, and plant products in a variety of ways. For example, the compositions can be used in a method that involves placing an effective amount of the pesticidal composition in the environment of the pest by a procedure selected from the group consisting of spraying, dusting, broadcasting, or seed coating.

Before plant propagation material (fruit, tuber, bulb, corm, grains, seed), but especially seed, is sold as a commercial product, it is customarily treated with a protectant coating comprising herbicides, insecticides, fungicides, bactericides, nematicides, molluscicides, or mixtures of several of these preparations, if desired together with further carriers, surfactants, or application-promoting adjuvants customarily employed in the art of formulation to provide protection against damage caused by bacterial, fungal, or animal pests. In order to treat the seed, the protectant coating may be applied to the seeds either by impregnating the tubers or grains with a liquid formulation or by coating them with a combined wet or dry formulation. In addition, in special cases, other methods of application to plants are possible, e.g., treatment directed at the buds or the fruit.

The plant seed of the embodiments comprising a nucleotide sequence encoding a pesticidal protein of the embodiments may be treated with a seed protectant coating comprising a seed treatment compound, such as, for example, captan, carboxin, thiram, methalaxyl, pirimiphos-methyl, and others that are commonly used in seed treatment. In one embodiment, a seed protectant coating comprising a pesticidal composition of the embodiments is used alone or in combination with one of the seed protectant coatings customarily used in seed treatment. The compositions of the embodiments can be in a suitable form for direct application or as a concentrate of primary composition that requires dilution with a suitable quantity of water or other diluent before application. The pesticidal concentration will vary depending upon the nature of the particular formulation, specifically, whether it is a concentrate or to be used directly. The composition contains 1 to 98% of a solid or liquid inert carrier, and 0 to 50% or 0.1 to 50% of a surfactant. These compositions will be administered at the labeled rate for the commercial product, for example, about 0.01 lb-5.0 lb. per acre when in dry form and at about 0.01 pts.-10 pts. per acre when in liquid form.

The codon optimized synthetic nucleotide sequence disclosed herein may also be used for production of transformed Bacillus thuringiensis capable of producing Cry2Ai protein. The transformed Bacillus thuringiensis may then be used for production of insecticidal and/or pesticidal composition useful for agricultural activity such as insect pest management.

In the embodiments, a transformed microorganism (which includes whole organisms, cells, spore(s) such as Bacillus thuringiensis transformed with the codon optimized synthetic nucleotide sequences disclosed herein, pesticidal protein(s), pesticidal component(s), pest-affecting component(s), mutant(s), living or dead cells and cell components, including mixtures of living and dead cells and cell components, and including broken cells and cell components) or an isolated pesticidal protein can be formulated with an acceptable carrier into a pesticidal composition(s) that is, for example, a suspension, a solution, an emulsion, a dusting powder, a dispersible granule or pellet, a wettable powder, and an emulsifiable concentrate, an aerosol or spray, an impregnated granule, an adjuvant, a coatable paste, a colloid, and also encapsulations in, for example, polymer substances. Such formulated compositions may be prepared by such conventional means as desiccation, lyophilization, homogenization, extraction, filtration, centrifugation, sedimentation, or concentration of a culture of cells comprising the polypeptide.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence plant growth. One or more agrochemicals including, but not limited to, herbicides, insecticides, fungicides, bactericides, nematicides, molluscicides, acaricides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the embodiments are normally applied in the form of compositions and can be applied to the crop area, plant, or seed to be treated. For example, the compositions of the embodiments may be applied to grain in preparation for or during storage in a grain bin or silo, etc. The compositions of the embodiments may be applied simultaneously or in succession with other compounds. Methods of applying an active ingredient of the embodiments or an agrochemical composition of the embodiments that contains at least one of the pesticidal proteins produced by the bacterial strains of the embodiments include, but are not limited to, foliar application, seed coating, and soil application. The number of applications and the rate of application depend on the intensity of infestation by the corresponding pest.

In a further embodiment, the compositions, as well as the transformed microorganisms and pesticidal protein of the embodiments, can be treated prior to formulation to prolong the pesticidal activity when applied to the environment of a target pest as long as the pre-treatment is not deleterious to the pesticidal activity. Such treatment can be by chemical and/or physical means as long as the treatment does not deleteriously affect the properties of the composition(s). Examples of chemical reagents include but are not limited to halogenating agents; aldehydes such as formaldehyde and glutaraldehyde; anti-infectives, such as zephiran chloride; alcohols, such as isopropanol and ethanol.

In other embodiments, it may be advantageous to treat the Cry toxin polypeptides with a protease, for example trypsin, to activate the protein prior to application of a pesticidal protein composition of the embodiments to the environment of the target pest. Methods for the activation of protoxin by a serine protease are well known in the art.

The compositions (including the transformed microorganisms and pesticidal protein of the embodiments) can be applied to the environment of an insect pest by, for example, spraying, atomizing, dusting, scattering, coating or pouring, introducing into or on the soil, introducing into irrigation water, by seed treatment or general application or dusting at the time when the pest has begun to appear or before the appearance of pests as a protective measure. For example, the pesticidal protein and/or transformed microorganisms of the embodiments may be mixed with grain to protect the grain during storage. It is generally important to obtain good control of pests in the early stages of plant growth, as this is the time when the plant can be most severely damaged. The compositions of the embodiments can conveniently contain another insecticide if this is thought necessary. In one embodiment, the composition is applied directly to the soil, at a time of planting, in granular form of a composition of a carrier and dead cells of a Bacillus strain or transformed microorganism of the embodiments. Another embodiment is a granular form of a composition comprising an agrochemical such as, for example, an herbicide, an insecticide, a fertilizer, an inert carrier, and dead cells of a Bacillus strain or transformed microorganism of the embodiments.

Those skilled in the art will recognize that not all compounds are equally effective against all pests. Compounds of the embodiments display activity against insect pests, which may include economically important agronomic, forest, greenhouse, nursery, ornamentals, food and fiber, public and animal health, domestic and commercial structure, household, and stored product pests.

The insect pests include insects from the order Lepidoptera, diptera, coleopteran, hemiptera and homoptera.

Insect pests of the order Lepidoptera include, but are not limited to, armyworms, cutworms, loopers, and heliothines in the family Noctuidae Spodoptera frugiperda J E Smith (fall armyworm); S. exigua Hubner (beet armyworm); S. litura Fabricius (tobacco cutworm, cluster caterpillar); Mamestra configurata Walker (bertha armyworm); M. brassicae Linnaeus (cabbage moth); Agrotis ipsilon Hufnagel (black cutworm); A. orthogonia Morrison (western cutworm); A. subterranea Fabricius (granulate cutworm); Alabama argillacea Hubner (cotton leaf worm); Trichoplusia ni Hubner (cabbage looper); Pseudoplusia includens Walker (soybean looper); Anticarsia gemmatalis Hubner (velvetbean caterpillar); Hypena scabra Fabricius (green cloverworm); Heliothis virescens Fabricius (tobacco budworm); Pseudaletia unipuncta Haworth (armyworm); Athetis mindara Barnes and Mcdunnough (rough skinned cutworm); Euxoa messoria Harris (darksided cutworm); Earias insulana Boisduval (spiny bollworm); E. vittella Fabricius (spotted bollworm); Helicoverpa armigera Hubner (American bollworm); H. zea Boddie (corn earworm or cotton bollworm); Melanchra picta Harris (zebra caterpillar); Egira (Xylomyges) curialis Grote (citrus cutworm); borers, casebearers, webworms, coneworms, and skeletonizers from the family Pyralidae Ostrinia nubilalis Hubner (European corn borer); Amyelois transitella Walker (naval orangeworm); Anagasta kuehniella Zeller (Mediterranean flour moth); Cadra cautella Walker (almond moth); Chilo suppressalis Walker (rice stem borer); C. partellus, (sorghum borer); Corcyra cephalonica Stainton (rice moth); Crambus caliginosellus Clemens (corn root webworm); C. teterrellus Zincken (bluegrass webworm); Cnaphalocrocis medinalis Guenee (rice leaf roller); Desmia funeralis Hubner (grape leaffolder); Diaphania hyalinata Linnaeus (melon worm); D. nitidalis Stoll (pickleworm); Diatraea grandiosella Dyar (southwestern corn borer), D. saccharalis Fabricius (surgarcane borer); Eoreuma loftini Dyar (Mexican rice borer); Ephestia elutella Hubner (tobacco (cacao) moth); Galleria mellonella Linnaeus (greater wax moth); Herpetogramma licarsisalis Walker (sod webworm); Homoeosoma electellum Hulst (sunflower moth); Elasmopalpus lignosellus Zeller (lesser cornstalk borer); Achroia grisella Fabricius (lesser wax moth); Loxostege sticticalis Linnaeus (beet webworm); Orthaga thyrisalis Walker (tea tree web moth); Maruca testulalis Geyer (bean pod borer); Plodia interpunctella Hubner (Indian meal moth); Scirpophaga incertulas Walker (yellow stem borer); Udea rubigalis Guenee (celery leaftier); and leafrollers, budworms, seed worms, and fruit worms in the family Tortricidae Acleris gloverana Walsingham (Western blackheaded budworm); A. variana Fernald (Eastern blackheaded budworm); Archips argyrospila Walker (fruit tree leaf roller); A. rosana Linnaeus (European leaf roller); and other Archips species, Adoxophyes orana Fischer von Rosslerstamm (summer fruit tortrix moth); Cochylis hospes Walsingham (banded sunflower moth); Cydia latiferreana Walsingham (filbertworm); C. pomonella Linnaeus (codling moth); Platynota flavedana Clemens (variegated leafroller); P. stultana Walsingham (omnivorous leafroller); Lobesia botrana Denis & Schiffermuller (European grape vine moth); Spilonota ocellana Denis & Schiffermuller (eyespotted bud moth); Endopiza viteana Clemens (grape berry moth); Eupoecilia ambiguella Hubner (vine moth); Bonagota salubricola Meyrick (Brazilian apple leafroller); Grapholita molesta Busck (oriental fruit moth); Suleima helianthana Riley (sunflower bud moth); Argyrotaenia spp.; Choristoneura spp.

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guerin-Meneville (Chinese Oak Silkmoth); Bombyx mori Linnaeus (Silkworm); Bucculatrix thurberiella Busck (cotton leaf perforator); Collas eurytheme Boisduval (alfalfa caterpillar); Datana integerrima Grote & Robinson (walnut caterpillar); Dendrolimus sibiricus Tschetwerikov (Siberian silk moth), Ennomos subsignaria Hubner (elm spanworm); Erannis tiliaria Harris (linden looper); Euproctis chtysorrhoea Linnaeus (browntail moth); Harrisina americana Guerin-Meneville (grapeleaf skeletonizer); Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Manduca quinquemaculata Haworth (five spotted hawk moth, tomato hornworm); M. sexta Haworth (tomato hornworm, tobacco hornworm); Operophtera brumata Linnaeus (winter moth); Paleacrita vemata Peck (spring cankerworm); Papilio cresphontes Cramer (giant swallowtail, orange dog); Phryganidia californica Packard (California oakworm); Phyllocnistis citrella Stainton (citrus leafminer); Phyllonotycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenee (omnivorous looper); Schizura concinna J. E. Smith (red humped caterpillar); Sitotroga cerealella Olivier (Angoumois grain moth); Thaumetopoea pityocampa Schiffermuller (pine processionary caterpillar); Tineola bisselliella Hummel (webbing clothesmoth); Tuta absolute Meyrick (tomato leafminer); Yponomeuta padella Linnaeus (ermine moth); Heliothis subflexa Guenee; Malacosoma spp. and Orgyia spp.

Insect pests may be tested for pesticidal activity of compositions of the embodiments in early developmental stages, e.g., as larvae or other immature forms. The insects may be reared in total darkness at from about 20° C. to about 30° C. and from about 30% to about 70% relative humidity. Methods of rearing insect larvae and performing bioassays are well known to one of ordinary skill in the art.

A method for controlling insects, particularly Lepidoptera, in accordance with this invention can comprise applying (e.g., spraying) the insecticidal/pesticidal composition disclosed herein to an area or plant to be protected to a locus (area) to be protected, comprising host cells transformed with the codon optimized cry2Ai nucleotide sequences of the invention. The locus to be protected can include, for example, the habitat of the insect pests or growing vegetation or an area where vegetation is to be grown.

The present disclosure relates to a method for controlling eggplant insect pests, which method comprises applying the insecticidal/pesticidal composition disclosed herein to an area or plant to be protected, by planting eggplant plants transformed with a cry2Ai nucleotide sequence(s) of the invention, or spraying a composition containing a Cry2Ai protein of the invention. The invention also relates to use of the composition of the invention against Lepidopteran eggplant insect pests to minimize damage to eggplant plants.

The present disclosure also relates to a method for controlling rice insect pests, such as Lepidopteran rice stemborers, rice leaffolders rice skippers, rice cutworms, rice armyworms, or rice caseworms, preferably an insect selected from the group consisting of: Chilo suppressalis, Chilo partellus, Scirpophaga incertulas, Sesamia inferens, Cnaphalocrocis medinalis, Marasmia patnalis, Marasmia exigua, Marasmia ruralis, Scirpophaga innotata, which method comprises applying the insecticidal/pesticidal composition disclosed herein to an area or plant to be protected to an area or plant to be protected, by planting a rice plant transformed with a cry2Ai nucleotide sequence(s) of the invention, or spraying a composition containing a Cry2Ai protein of the invention. The invention also relates to use of the composition disclosed herein, against rice insect pests to minimize damage to rice plants.

The present disclosure further relates to a method for controlling tomato insect pests, such as Lepidopteran Helicoverpa armigera which method comprises applying the insecticidal/pesticidal composition disclosed herein to an area or plant to be protected to an area or plant to be protected, by planting a tomato plant transformed with a cry2Ai nucleotide sequence(s) of the invention, or spraying a composition containing a Cry2Ai protein of the invention. The invention also relates to use of the composition disclosed herein, against tomato insect pests to minimize damage to tomato plants.

The present disclosure further relates to a method for controlling cotton insect pests, which method comprises applying the insecticidal/pesticidal composition disclosed herein to an area or plant to be protected to an area or plant to be protected, by planting a cotton plant transformed with a cry2Ai nucleotide sequence(s) of the invention, or spraying a composition containing a Cry2Ai protein of the invention. The invention also relates to use of the composition disclosed herein, against cotton insect pests to minimize damage to cotton plants.

To obtain the Cry2Ai toxin protein (SEQ ID NO:1), cells of the recombinant hosts expressing the Cry2Ai protein can be grown in a conventional manner on a suitable culture medium and then lysed using conventional means such as enzymatic degradation or detergents or the like. The toxin can then be separated and purified by standard techniques such as chromatography, extraction, electrophoresis, or the like.

Accordingly, the present invention provides compositions and methods for affecting insect pests, particularly plant pests. More specifically, the invention provides codon optimised synthetic nucleotide sequence that encodes biologically active insecticidal polypeptides against insect pests such as, but not limited to, insect pests of the order Lepidopteran pests, such as Helicoverpa armigera—the cotton bollworm and corn earworm, Cnaphalocrocis medinalis—the rice leaffolder, and Scirpophaga incertulas—the rice yellow stem borer, and Spectinophora gossypiella.

In accordance with the present disclosure, in one of the embodiment there is provided a codon optimized synthetic nucleotide sequence encoding protein having amino acid sequence as set forth in SEQ ID NO: 1, wherein said nucleotide sequence is selected from a group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and (b).

In another embodiment, the present invention provides a nucleic acid molecule comprising a codon optimized sequence for expression in a plant selected from the group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and b).

In one of the embodiment there is provided a codon optimized synthetic nucleotide sequence encoding protein having amino acid sequence as set forth in SEQ ID NO: 1, wherein said nucleotide sequence is
a. as set forth in SEQ ID NO: 2, or a nucleotide sequence complementary thereto; or
b. a nucleotide sequence which specifically hybridizes to at least 10 nucleotides of the nucleotide sequence as set forth in SEQ ID NO: 2 from nucleotide position 262 to 402 and/or 1471 to 1631 or a nucleotide sequence complementary thereto

In another embodiment there is provided the codon optimized synthetic nucleotide sequence of the present disclosure, wherein the nucleotide sequence which specifically hybridizes to the nucleotide sequence as set forth in SEQ ID NO: 2 is selected from the group consisting of SEQ ID NO: 3, SEQ ID NO: 4, SEQ ID NO: 5 and SEQ ID NO: 6.

In another embodiment there is provided a recombinant DNA comprising the codon optimized synthetic nucleotide sequence of the present disclosure, wherein the nucleotide sequence is operably linked to a heterologous regulatory element. Another embodiment relates to the recombinant DNA as disclosed herein, wherein said codon optimized synthetic nucleotide sequence optionally comprises a selectable marker gene, a reporter gene or a combination thereof. Yet another embodiment related to the recombinant DNA as disclosed herein, wherein said codon optimized synthetic nucleotide sequence optionally comprises a DNA sequence encoding a targeting or transit peptide for targeting to the vacuole, mitochondrium, chloroplast, plastid, or for secretion.

In another embodiment there is proved a DNA construct for expression of an insecticidal protein of interest comprising a 5′ non-translated sequence, a coding sequence encoding an insecticidal Cry2Ai protein comprising the amino acid sequence of SEQ ID NO: 1 or an insecticidal portion thereof, and a 3′ non-translated region, wherein said 5′ non-translated sequence comprises a promoter functional in a plant cell, said coding sequence is a codon optimized synthetic nucleotide sequence as disclosed herein, and wherein said 3′ non-translated sequence comprises a transcription termination sequence and a polyadenylation signal.

One of the embodiments is related to a plasmid vector comprising the codon optimized synthetic nucleotide sequence as disclosed herein. Another embodiment provides a plasmid vector comprising the recombinant DNA comprising the codon optimized synthetic nucleotide sequence as disclosed herein. Further embodiment provides a plasmid vector comprising the DNA construct comprising the codon optimized synthetic nucleotide sequence as disclosed herein.

Another embodiment provides a host cell comprising the codon optimized synthetic nucleotide sequence of the present disclosure. The host cell of the present disclosure encompasses a plant, bacterial, virus, fungi and a yeast cell. In another embodiment the host cell as disclosed is a plant cell, Agrobacterium or E. coli.

Another embodiment of the present invention provides a method for conferring an insect resistance in a plant comprising
(a) inserting into a plant cell a codon optimized synthetic nucleotide sequence as disclosed herein, wherein the nucleotide sequence is operably linked to a (i) promoter functional in a plant cell and (ii) a terminator;
(b) obtaining a transformed plant cell from the plant cell of step (a), wherein said transformed plant cell comprises the said codon optimized synthetic nucleotide sequence of the disclosure; and
(c) generating a transgenic plant from said transformed plant cell of step (b), wherein said transgenic plant comprises the said codon optimized synthetic nucleotide sequence of the disclosure.

Another embodiment relates to a transgenic plant obtained by the method for conferring an insect resistance as disclosed herein. Yet another embodiment of the present invention relates to a transgenic plant comprising the codon optimized synthetic nucleotide sequence as disclosed herein. Still another embodiment of the present invention relates to a transgenic plant comprising the codon optimized synthetic nucleotide sequence, wherein the nucleotide sequence is selected from the group consisting of SEQ ID NO: 2, SEQ ID NO:3, SEQ ID NO: 4, SEQ ID NO:5 and SEQ ID NO: 6. Yet another embodiment of the present disclosure relates to transgenic plant disclosed herein, wherein said plant is selected from a group consisting of cotton, eggplant, rice, wheat, corn, sorghum, oat, millet, legume, tomato, cabbage, cauliflower, broccoli, Brassica sp., beans, pea, pigeonpea, potato, pepper, cucurbit, lettuce, sweet potato canola, soybean, alfalfa, peanuts, sunflower, safflower, tobacco, sugarcane, cassava, coffee, pineapple, citrus, cocoa, tea, banana and melon.

Further embodiment of the present disclosure relates to the a tissue, seed or a progeny obtained from the transgenic plant of the present invention, wherein said seed or progeny comprises the codon optimized synthetic nucleotide sequence as disclosed herein. Yet another embodiment of the present invention relates to a biological sample derived from the tissues or seed or progeny as disclosed herein, wherein said sample comprising a detectable amount of said codon optimized synthetic nucleotide sequence of the present invention. Further embodiment of the present invention provides a commodity product derived from the transgenic plant disclosed in the present disclosure, wherein said product comprises a detectable amount of said codon optimized synthetic nucleotide sequence as disclosed herein.

Another embodiment of the present invention provides a composition comprising Bacillus thuringiensis comprising the codon optimized synthetic nucleotide sequence of the present disclosure encoding Cry2Ai protein having amino acid sequence as set forth in SEQ ID NO: 1. The composition as disclosed herein may optionally comprises an additional insecticidal agent toxic to same insect pest but exhibiting a different mode of its insecticidal activity from said insecticidal protein. The insecticidal agent of the composition of the disclosure is selected from the group consisting of a Bacillus toxin, a Xenorhabdus toxin, a Photorhabdus toxin, and a dsRNA specific for suppression of one or more essential genes in said insect pest.

Yet another embodiment of the present invention provides a method of controlling insect infestation in a crop plant and providing insect resistance management, wherein said method comprising contacting said crop plant with a insecticidally effective amount of the composition as described above.

Further embodiment of the present invention relates to use of the codon optimized synthetic nucleotide sequence, the DNA construct or the plasmid of the disclosure for production of insect resistant transgenic plants. Yet another embodiment of the present invention relates to use of the codon optimized synthetic nucleotide sequence as disclosed herein for production of insecticidal composition, wherein the composition comprises Bacillus thuringiensis cells comprising the said nucleotide sequences.

Another embodiment of the present invention provides transgenic plants which express at least one codon optimized synthetic nucleotide sequence disclosed herein. Further embodiment provides a transgenic plant obtained by the method disclosed herein. The transgenic plant disclosed herein is selected from a group consisting of rice, wheat, corn, sorghum, oat, millet, legume, cotton, tomato, eggplant, cabbage, cauliflower, broccoli, Brassica sp., beans, pea, pigeonpea, potato, pepper, cucurbit, lettuce, sweet potato canola, soybean, alfalfa, peanuts, sunflower, safflower, tobacco, sugarcane, cassava, coffee, pineapple, citrus, cocoa, tea, banana and melon. Some embodiments of the invention relate to tissue, seeds or a progenies obtained from the transgenic plant(s) of the invention, wherein said seed or progeny comprises the codon optimized synthetic nucleotide sequence described herein. Some embodiments of the invention provide biological samples derived from the tissues or seeds or progenies, wherein the sample comprises a detectable amount of said codon optimized synthetic nucleotide sequence. One embodiment encompasses a commodity product derived from the transgenic plant of the invention, wherein the product comprises a detectable amount of the codon optimized synthetic nucleotide sequence.

In one embodiment there is provides a composition comprising Bacillus thuringiensis comprising at least one codon optimized synthetic nucleotide sequence of the disclosure, wherein the nucleotide sequence encodes Cry2Ai protein having amino acid sequence as set forth in SEQ ID NO: 1. The composition optionally comprises an additional insecticidal agent toxic to same insect pest but exhibiting a different mode of its insecticidal activity from said insecticidal protein. The insecticidal agent is selected from the group consisting of a Bacillus toxin, a Xenorhabdus toxin, a Photorhabdus toxin, and a dsRNA specific for suppression of one or more essential genes in said insect pest.

In another embodiment there is provided a method of controlling insect infestation in a crop plant and providing insect resistance management, wherein said method comprising contacting said crop plant with an insecticidally effective amount of the composition described herein.

Another embodiment relates to use of the codon optimized synthetic nucleotide sequence, the DNA construct or the plasmid of the disclosure for production of insect resistant transgenic plants. Yet another embodiment relates to use of the codon optimized synthetic nucleotide sequence disclosed herein for production of insecticidal composition, wherein the composition comprises Bacillus thuringiensis cells comprising the said nucleotide sequences.

These and/or other embodiments of this invention are reflected in the wordings of the claims that form part of the description of the invention.

Various modifications and other embodiments of the present invention can be presented by a person skilled in the art to which these inventions pertain having the benefit of the teachings presented in the descriptions and the associated drawings. Therefore, it is to be understood that the present invention is not to be limited to the specific embodiments disclosed herein and that modifications and other embodiments are intended to be included within the scope of the appended claims.

The following Examples illustrate the invention, and are not provided to limit the invention or the protection sought.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperatures, etc.) but some experimental errors and deviations should be accounted for.

### General Methods

DNA manipulations were done using procedures that are standard in the art. These procedures can often be modified and/or substituted without substantively changing the result. Except where other references are identified, most of these procedures are described in Sambrook et al., Molecular Cloning: A Laboratory Manual, Cold Spring Harbor Laboratory Press, second edition, 1989.

### Example 1: Designing a Codon Optimized Sequence Encoding Cry2Ai Protein (SEQ ID NO: 1)

A DNA sequence having plant codon bias was designed and synthesized for expression of the Cry2Ai protein having amino acid sequence as set forth in SEQ ID NO: 1 in transgenic plants. A codon usage table for plant was calculated from coding sequences obtained from the Cry2Ai protein sequence of SEQ ID NO: 1 (NCBI GenBank ACV97158.1). The DNA sequence was optimised using Monte Carlo algorithm (Villalobos A, Ness J E, Gustafsson C, Minshull J and Govindarajan S (2006) Gene Designer: a synthetic biology tool for constructing artificial DNA segments; BMC Bioinformatics 67:285-293) as the primary criteria for selection of codons. Probabilities from codon usage table were considered while optimising the DNA sequence. Rare and less frequent codons were replaced by most abundant codons. A weighted-average plant codon set was calculated after omitting any redundant codon used less than about 10% of total codon uses for that amino acid. The Weighted Average representation for each codon was calculated using the formula:

Weighted Average % of CI=1/(% C1+% C2+% C3+ etc.) x % C1×100 where CI is the codon in question and % C2, % C3, etc. represent the average % usage values of the remaining synonymous codons.

To derive plant-codon-optimized DNA sequence encoding the Cry2Ai protein of SEQ ID NO: 1, codon substitutions in the DNA sequence encoding the Cry2Ai protein were made such that the resulting DNA sequence had the overall codon composition of plant optimized codon bias table. Further refinements to the sequences were made to eliminate undesirable restriction enzyme recognition sites, potential plant intron splice sites, long runs of A/T or C/G residues, and other motifs that might interfere with RNA stability, transcription, or translation of the coding region in plant cells. The gene was optimized in-silico by using gene designer software with 6.0 kcal/mole cut-off value for formation of stable secondary structure of mRNA. Other changes were made to incorporate desired restriction enzyme recognition sites, and to eliminate long internal Open Reading Frames (frames other than +1). Restriction recognition sites of XbaI, NcoI and BamHI were incorporated before start codon ATG and restriction recognition site SmaI was inserted before stop codon to enable cloning of the optimized gene in prokaryotic vector for fusion of C terminal protein tags. Restriction recognition sites of EcoRI and HindIII were incorporated after Stop codon. Stop codon TGA was used in the optimized gene.

These changes were all made within the constraints of retaining approximately plant biased codon composition. A complete plant-codon-optimized sequence encoding the Cry2Ai protein (SEQ ID NO: 1) is as set forth in SEQ ID NOs: 2-6. In-silico translation of plant-biased codon optimized DNA sequence showed 100% identity with the native Cry2Ai protein (SEQ ID NO: 1). The plant-codon-optimized DNA sequences (SEQ ID NOs: 2-6) were designated as 201D1, 201D2, 201D3, 201D4 and 201D5. Synthesis of the 201D1-D5 DNA fragments (SEQ ID NOs: 2-6) was performed by a commercial vendor (Genscript Inc, USA). The 201D1DNA fragment was cloned in pUC57 vector by using the method known in the art and designated as pUC57-201D1. Similarly 201D2 DNA, 201D3 DNA, 201D4 DNA and 201D5 DNA fragments were cloned in pUC57 vector and designated as pUC57-201D2, pUC57-201D3, pUC57-201D4 and pUC57-201D5, respectively.

### Example 2: Construction of Expression Vector

The pUC57-201D1 vector of Example 1 was digested with BamHI and HindIII to obtain 201D1 DNA (SEQ ID NO: 2) fragment (Reaction volume—20 μl, Plasmid DNA—8.0 μl, 10× buffer—2.0 μl, Restriction enzyme BamHI—0.5 μl, Restriction enzyme HindIII—0.5 μl, Distilled water—9.0 μl). All the reagents were mixed to obtain reaction mixture and incubated at 37° C. for 30 mins subsequently the reaction mixture was analysed by gel electrophoresis on 2% agarose gel and the 201D1 DNA fragment was excised from the agarose gel with a clean sharp scalpel, under UV illumination. The DNA fragment was eluted from gel using the gel elution kit. The gel slice containing the DNA fragment was transferred into a 2 ml eppendorf tube and 3× sample volume of buffer DE-A was added. The gel was re-suspended in buffer DE-A by vortexing and contents were heated to 75° C. until the gel was completely dissolved, followed by addition of 0.5× buffer DE-A and DE-B mixed together. An eppendorf tube was prepared by placing a column into it, and binding mix was transferred to the column. The eppendorf tube with column was centrifuged briefly. The column was placed into a fresh eppendorf tube and 500 μl of buffer washing buffer—W I (Qiagen Kit) was added followed by centrifugation. The supernatant was discarded and 700 μl of washing buffer—W 2 was added along the walls of column to wash off all residual buffer followed by centrifugation. This step was repeated with 700 μl aliquot of the buffer W 2. The column was transferred to a fresh eppendorf tube and centrifuged at 6000 rpm for 1 min to remove the residual buffer. The column was again placed in a new eppendorf tube and 40 μl of eluent buffer was added at the centre of the membrane. The column with eluent buffer was allowed to stand for 1 minute at room temperature and tubes were centrifuged at 12000 rpm for 1 min. The eluted 201D1 DNA fragment was stored at −20° C. until further use.

The isolated and purified 201D1 DNA fragment thus obtained was ligated in linearized pET32a vector. The ligation was carried out using T4DNA ligase enzyme (Reaction volume—30 μl, 10× ligation buffer—3.0 μl, Vector DNA—5.0 μl, Insert DNA—15.0 μl, T4 DNA Ligase enzyme—1.0 μl, Distilled water −6.0 μl). The reagents were mixed well and the resulting reaction mixture was incubated at 16° C. for 2 hours. Subsequently competent cells of E. coli BL21 (DE3) strain were transformed with the ligation mixture comprising the pET32a vector carrying the 201D1 DNA by adding 100 of ligation mixture to 100 μl of BL21 DE3 competent cells. The cell mixture thus obtained was placed on ice for 30 mins and incubated at 42° C. for 60 sec in a water bath for heat shock and placed back to on ice for 5-10 mins. Subsequently 1 ml LB broth was added to the mixture and incubated further at 37° C. for 1 hour in incubator shaker at 200 rpm. The cell mixture was spread on LB agar plus 50 μg/ml carbenicillin and incubated at 37° C. overnight. Positive clones were identified by restriction digestion analysis. The expression vector thus obtained was designated as pET32a-201D1.

Similarly expression vectors carrying the other plant codon optimized DNA sequences as set forth in SEQ ID NO: 3-6 were constructed and were designated as pET32a-201D2, pET32a-201D3, pET32a-201D4, and pET32a-201D5.

### Example 3: Expression of CRY2Ai in E coli

The 201D1 DNA (SEQ ID NO: 2) cloned in pET32a, designated as pET32a-201D1 was expressed in E. coli BL 21 D3. The expression was induced with 1 mM IPTG at a cell density of about OD nm=0.5 to 1.0. After the induction, the E. coli cells were incubated in a shaker for 24 to 40 hours at 16° C. for protein production. The Cry2Ai protein (SEQ ID NO: 1) was expressed in a soluble form in the cell. Subsequently the culture was transferred to a centrifuge tube and centrifuged at 10000 rpm for 5 min. The supernatant was discarded and 10 ml of the cell were digested with lysozyme and incubated for 1 hr at room temperature. The samples were centrifuged at 10000 rpm for 5 mins and supernatant was discarded. The pellet was suspended in sterile distilled water and centrifuged at 10000 rpm for 10 mins. This step was repeated twice and the pellet was stored at −20° C. The pellet containing proteins from the induced recombinant strain were analysed on 10% SDS-PAGE.

Similarly the DNA sequences as set forth in SEQ ID NO: 3-6 were cloned in pET32a, and expressed in E. coli BL 21 D3.

### Example 4: Construction of Plant Transformation Vector Containing 201D1 DNA (SEQ ID NO: 2) Encoding Cry2Ai Protein (SEQ ID NO: 1)

The pUC57-201D1 vector carrying 201D1 DNA (SEQ ID NO: 2) was digested with restriction enzymes to release the 201D1 DNA fragment (Reaction volume—200, Plasmid DNA—8.0 μl, 10× buffer—2.0 μl, Restriction enzyme EcoRV—0.5 μl, Distilled water—9.5 μl). All the reagents were mixed and the mixture was incubated at 37° C. for 30 mins. The product obtained after the restriction digestion was analysed by gel electrophoresis and further purified further.

The Ti plasmid pGreen0029 vector was prepared by restriction digestion with

EcoRV enzyme (Reaction volume—200, Plasmid DNA—8.0 μl, 10× buffer −2.0 μl, Restriction enzyme EcoRV—0.5 μl, Distilled water—9.5 μl). All the reagents were mixed and the mixture was incubated at 37° C. for 30 mins. The product obtained after the restriction digestion was analysed by gel electrophoresis.

The purified 201D1 DNA fragment (SEQ ID NO: 2) was ligated in linearized pGreen0029 vector. The ligation was carried out using T4DNA ligase enzyme (Reaction volume—30 μl, 10× ligation buffer—3.0 μl, Vector DNA—5.0 μl, Insert DNA—15.0 μl, T4 DNA Ligase enzyme—1.0 μl, Distilled water −6.0 μl). The reagents were mixed well and the resulting reaction mixture was incubated at 16° C. for 2 hours. Subsequently competent cells of E. coli BL21 (DE3) strain were transformed with the ligation mixture comprising the pGreen0029 vector carrying the 201D1 DNA (SEQ ID NO: 2) by adding 100 of ligation mixture to 100 μl of BL21 DE3 competent cells. The cell mixture thus obtained was placed on ice for 30 mins and incubated at 42° C. for 60 sec in a water bath for heat shock and placed the cell mixture back to ice for 5-10 mins. Subsequently 1 ml LB broth was added to the mixture and further incubated at 37° C. for 1 hour in incubator shaker at 200 rpm. The cell suspension (100 μl) was uniformly spread on LB agar medium containing 50 μg/ml carbenicillin. The plates were incubated at 37° C. overnight. The positive clones were confirmed by restriction digestion analysis. The recombinant vector thus obtained was designated as pGreen0029 CaMV35S-201D1.

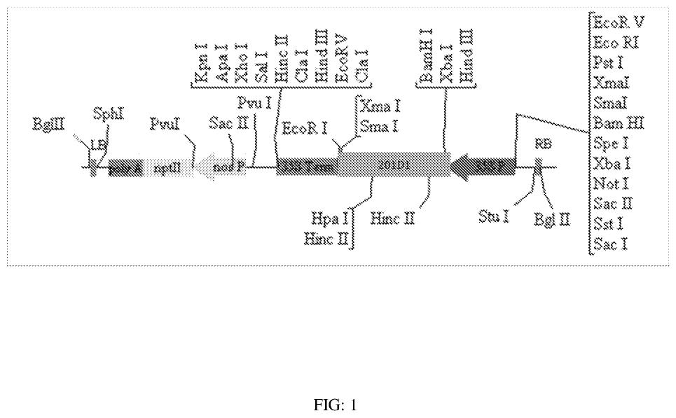

Thus, the recombinant plasmid pGreen0029-CaMV35S-201D1 contains the Plant-optimized 201D1 DNA sequence (SEQ ID NO: 2) under the transcriptional control of the 35SCaMV promoter. Further, pGreen0029-CaMV35S-201D1 contains nptII gene, a plant selectable marker gene under the transcriptional control of NOS promoter (FIG. 1). The physical arrangement of the components of the pGreen0029-CaMV35S-201D1 T-region is illustrated as:

RB>35SCaMV: 201D1 CDS: CaMV polyA>NOS promoter: nptII CDS: NOS Poly A>LB

Similarly the DNA sequences as set forth in SEQ ID NO: 3-6 were cloned in

The Ti plasmid pGreen0029 and the recombinant vectors thus obtained were designated as pGreen0029-CaMV35S-201D2, pGreen0029-CaMV35S-201D3, pGreen0029-CaMV35S-201D4, and pGreen0029-CaMV35S-201D5. The physical arrangement of the components of the pGreen0029-CaMV35S carrying the said DNA sequences (SEQ ID NO: 3-6) in the T-region is illustrated as:

RB>35SCaMV: 201D2 CDS: CaMV polyA>NOS promoter: nptII CDS: NOS Poly A>LB

RB>35SCaMV: 201D3 CDS: CaMV polyA>NOS promoter: nptII CDS: NOS Poly A>LB

RB>35SCaMV: 201D4 CDS: CaMV polyA>NOS promoter: nptII CDS: NOS Poly A>LB

RB>35SCaMV: 201D5 CDS: CaMV polyA>NOS promoter: nptII CDS: NOS Poly A>LB

Transformation of Agrobacterium tumefaciens with Recombinant Vector pGreen0029-CaMV35S-201D1

Agrobacterium tumefaciens strain LBA440 was transformed with the recombinant pGreen0029-CaMV35S-201D1 plasmid carrying the DNA sequence as set forth in SEQ ID NO: 2. 200 ng of the pGreen0029-CaMV35S-201D1 plasmid DNA was added to an aliquot of 100 μl of A. tumefaciens strain LBA440 competent cells. The mixture was incubated on ice for 30 min and transferred to liquid nitrogen for 20 mins followed by thawing at room temperature. The Agrobacterium cells were then transferred to 1 ml LB broth and incubated at 28° C. for 24 hours in water bath shaker at 200 rpm. The cell suspension was uniformly spread on LB agar medium containing 50 μg/ml rifampicin, 30 μg/ml kanamycin and 5 μg/ml tetracycline. The plates were incubated at 28° C. overnight. Transformed Agrobacterium cells were analysed plasmid extraction and restriction digestion method and positive A. tumefaciens colonies were selected and stored for further use.

### Example 5 (a) Agrobacterium-Mediated Cotton Transformation with pGreen0029-CaMV35S-201D1 Construct

Experimental details of cotton transformation are described below. Those skilled in the art of cotton transformation will understand that other methods are available for cotton transformation and for selection of transformed plants when other plant expressible selectable marker genes are used.

### Material

Agrobacterium tumefaciens Strains and Selectable Marker

tumefaciens LBA4404 and the neomycin phosphotransferase II (nptII) gene as a selectable marker have been used for cotton transformation and regeneration experiment described herein.

### Culture Media and Other

Luria-Bertani (LB) medium (Himedia); LB agar (Himedia); MS macro salts (Himedia), MS micro salts (Himedia), FeEDTA, B5 vitamins, thiamine-HCl (Duchefa), pyridoxine-HCl (Duchefa), nicotinic acid (Duchefa)], myo-inositol (Sigma), sucrose (Sigma), agar (Duchefa); 2, 4-Dichlorophenoxyacetic acid (2, 4-D) (Duchefa): 1 mg/mL stock; Kinetin (Duchefa); Indole-3-butyric acid/IBA (Duchefa); Acetosyringone (3′,5′-Dimethoxy-4′-hydroxyacetphenone) (Sigma); Augmentin (Duchefa); Kanamycin monosulphate (Duchefa);

### Plant Material

Cotton (Gossypium hirsutum) L. var Coker 310

### In Vitro Seed Germination and Pre-Culture

Cotton seeds var Coker 310 were immersed in sterile water with 0.1% Tween-20 in a shaker at 28° C.±2° C., 200 rpm for 20 min and treated with 0.1% HgCl₂ in the shaker for 20 min. The seeds were rinsed five times with sterile water. The sterilised seeds were soaked in sterile water overnight. The sterilized seeds were germinated in MS medium under photoperiod of 16/8 light/dark at 25° C.±2° C. The cotyledonary explants were prepared from 7 day old seedlings and precultured on MS medium comprising MS Salts, B5 vitamins, glucose: 30.0 g/1; Phytagel: 2.5 g/l pH: 5.8) with 2,4-D (1.0 mg/1) and kinetin (5.0 mg/1), with abaxial side touching the medium.

### Co-Cultivation, Selection and Plant Regeneration

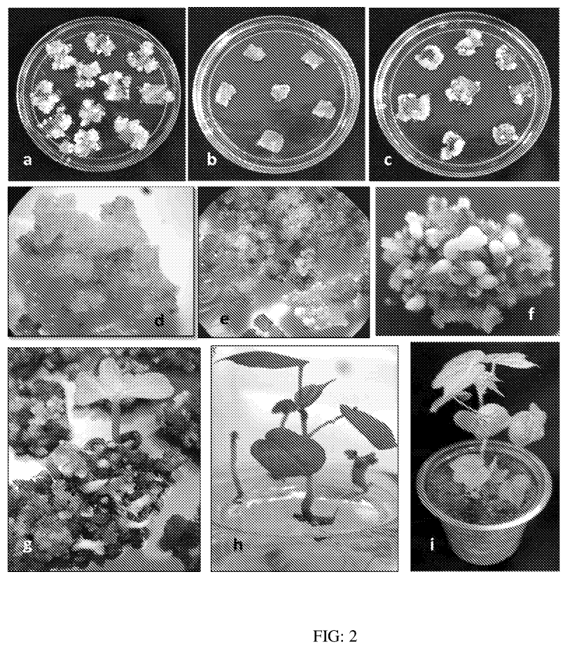

After 24 hours, the explants from the pre-culture medium were infected for 20 minutes with suspension culture of A. tumefaciens LBA4404 harbouring the plasmid pGreen0029-CaMV35S-201D1. The suspension culture medium was comprised of with 100 uM Acetosyringone. Excess suspension culture was removed by blot drying with sterile filter paper and transferred to co-cultivation medium comprising MS medium with 2,4-D (1.0 mg/1) and kinetin (5.0 mg/1), + acetosyringone (100 μM). After 48 hrs of co-cultivation in dark at 25° C.±2° C., the explants were washed with sterile distilled water and an aqueous solution containing 300 mg/l of Augmentin. The co-cultivated explants were blot dried on sterile filter paper and cultured on the selection medium comprising MS medium with 2,4-D (1.0 mg/1) and kinetin (5.0 mg/1), + kanamycin (50 mg/1)+Augmentin (300 mg/1). The explants were sub cultured on the same medium every two weeks on same medium till the calli appear. The calli was collected and sub-cultured on MS medium+kanamycin (50 mg/1)+Augmentin (300 mg/1). The proliferating calli were subcultured every 21 days on same medium. The embryogenic calli was identified and subcultured on MS medium with additional KNO₃ (1.9 g/1)+Augmentin (300 mg/1) to obtain somatic embryos. The somatic embryos appearing from embryogenic calli were further subcultured on MS medium+Augmentin (300 mg/1). The embryos along with adhering callus were placed on a filter paper resting on the culture media. The developed somatic embryos were then transferred to glass bottles containing half strength MS medium. The somatic embryos grew normally and turned into plantlets in 14-25 days. The plantlets were taken out carefully from tissue culture bottles and were hardened in small plastic pots containing soilrite and were maintained at 28±2° C. for 7-8 days. Subsequently the plantlets were transferred to greenhouse (FIG. 2).

Those skilled in the art of cotton transformation and regeneration will understand that other methods are available for cotton transformation, regeneration. Also for selection of transformed plants other plant expressible selectable marker genes can be used.

### Example 5 (B) Molecular Analyses of Putative Transgenic Cotton Plants Transformed with the 201D1 DNA Sequence (SEQ ID NO: 2)

### (i) Genomic DNA Isolation

Total genomic DNA was extracted from leaf tissues of the putative transgenic cotton plants obtained from Example 5 (A) and control non-transgenic plants of Coker 310. Leaves were collected from the putative transgenic cotton plants and non-transgenic cotton plants and were homogenized with 300 μl of extraction buffer (1M Tri s-HCl, pH 7.5, 1M NaCl, 200 mM EDTA and 10 percent SDS) using QIAGEN TissueLyser II (Retsch) and centrifuged at 12000 rpm for 10 minutes. The supernatant was transferred to a sterile microfuge tube. To the supernatant, chloroform-isoamyl alcohol (24:1) was added and centrifuged at 12000 rpm for 10 minutes. The aqueous layer was transferred to a microfuge tube. To that equal volume of ice-cold isopropanol was added and kept at −20° C. for 20 minutes. The supernatant was then discarded and to the pellet 300 μl of ethanol was added. After centrifugation at 10000 rpm for 5 minutes the supernatant was discarded and the DNA pellet was air dried for 15 minutes and subsequently dissolved in 40 μl of 0.1× TE buffer (Tris—pH 8.0: 10.0 mM and EDTA—pH 8.0: 1.0 mM). The quality and quantity of the genomic DNA was assessed using Nanodrop 1000® spectrophotometer (Thermo Scientific, USA) by measuring OD at 260 nm. Further, intactness of DNA was assessed by performing electrophoresis with 0.8 percent agarose gel.

### (ii) PCR Analysis

Polymerase chain reaction (PCR) was performed on the genomic DNA (100 ng) of the putative transgenic cotton plants and non-transgenic control cotton plants for analysis of synthetic cry2Ai-201D1 DNA using primers as set forth in SEQ ID NO: 7 and SEQ ID NO: 8 and nptII gene using primers as set forth in SEQ ID NO: 9 and SEQ ID NO: 10.

PCR conditions
94° C. for 5 min: 1 cycle
94° C. for 45 sec
58°−62° C. for 45 sec 30 cycles
72° C. for 45 sec
72° C. for 10 min: 1 cycle

All the putative transgenic cotton plants were found to be positive for the cry2Ai-201D1 DNA (1500 bp) having nucleotide sequence as set forth in SEQ ID NO: 2 and nptII gene (698 bp).

(iii) Southern Hybridization

All the PCR positive cotton plants were selected for southern hybridization. Genomic DNA (5 μg each) of ELISA positive T₀ transgenic cotton plants were digested with NcoI or XbaI or BamHI restriction enzymes and subjected to Southern blot hybridization using [α-32P]-dCTP labelled 201D1 DNA probe.

Genomic DNA of PCR and ELISA positive transgenic cotton plants and non-transgenic cotton plant was digested with NcoI restriction enzyme at 37° C. for 16 hours. The plasmid DNA pGreen0029-CaMV35S-201D1 was used as a positive control. The digested genomic DNA samples and plasmid DNA were resolved in 0.8% agarose gel at 20 V for overnight in 1×TAE buffer, visualized upon ethidium bromide staining under UV transilluminator and documented in gel documentation system (SYNGENE).

The restriction digested and electrophoretically separated genomic DNA was denatured by submerging the gels in two volumes of denaturing solution for 30 minutes with gentle agitation. The gels were subjected to neutralization by submerging it in two volumes of neutralizing solution for 30 minutes with gentle agitation. The gel was washed briefly in sterile de-ionized water and the DNA was transferred to positively charged nylon membrane (Sigma) through upward capillary transfer in 20×SSC buffer for 16 h following standard protocol. After complete transfer of genomic DNA, the nylon membrane was washed briefly in 2×SSC buffer and air dried for 5 minutes. The DNA was cross-linked by exposing the membrane in UV-cross linker (UV Stratalinker® 1800 Stratagene, CA, USA) at 1100 μJ for 1 minute. The cross-linked membrane was sealed in plastic bags and kept at 4° C. until used for Southern blot hybridization.

Denaturation Solution:
NaCl: 1M
NaOH: 0.5N

Neutralization Solution:
NaCl: 1.5M
Tris: 0.5M

20×SSC:
NaCl: 3.0M
Sodium citrate: 0.3M
pH: 7.0 with conc.HCl
   The 20×SSC Solution can be diluted as follows:

| Final SSC concentration | 20x SSC Solution | distilled or deionised water |
|---|---|---|
| 3x SSC | 30 ml | 170 ml |
| 2x SSC | 20 ml | 180 ml |
| 1x SSC | 10 ml | 190 ml |
| 0.5x SSC | 5 ml | 195 ml |

### Preparation of Probe

About 100 ng of 1500 bp of 201D1 DNA fragment amplified from the vector and purified using gel extraction miniprep kit (Bio Basic Inc., Canada) was radiolabelled with [α-32P]-dCTP was used as a labelling probe.

Probe labelling: Four μl of the PCR amplified and purified was used as template DNA for labelling. The template DNA was mixed with 10 μl of random primer (DecaLabel DNA Labeling Kit, Thermo Fisher Scientific Inc. USA) in a microfuge tube. The volume was made upto 40 μl with sterile distilled water and denatured by heating for 5 minutes on boiling water bath and cooled on ice. To the denatured DNA, 5 μl of labeling mix containing dATP, dGTP, dTTP, 5 μl of [α-32P]-dCTP (50 μCi) and 1 μl of Klenow fragment of DNA polymerase I was added and incubated at 37° C. for 10 minutes in a water bath. The reaction was stopped by adding 1 μl of 0.5M EDTA and incubated in a boiling water bath for 4 minutes and transferred onto ice for 4-5 minutes.

Pre-Hybridization and Hybridization with Probe

The DNA cross linked membrane as described above was gently placed into the hybridization bottle containing 30 ml of hybridization buffer solution. The bottle was tightly closed and placed in oven at 65° C. for 45 minutes to 1 hour for pre-hybridization treatment.

The hybridization buffer was poured off from the bottle and replaced with 30 ml of hybridization solution (maintained at 65° C.) containing denatured [α-32P]-dCTP labelled DNA probe. The bottle with hybridization solution was closed tightly and placed in the oven at 65° C. for 16 h.

Hybridization Solution:
Na2HPO4, pH 7.2: 0.5M
SDS: 7% (W/V)
EDTA, pH 7.2: 1 mM

The hybridization buffer was poured off from the bottle. About Wash I solution was added and the bottle was placed in oven on a slowly rotating platform at 65° C. for 10 minutes. After 10 minutes, the Wash I solution was replaced with 30 ml of Wash II solution and incubated at 65° C. for 5-10 min with gentle agitation. Then the Wash II solution was poured off and radioactivity count in the membrane was checked using Gregor-Muller counter. Depending on count, 30 ml of Wash III was added to the bottle and incubated at 65° C. for 30 seconds to 1 minute with gentle agitation. Subsequently Wash III solution was removed and the membrane was dried on Whatman No. 1 filter paper for 5-10 minutes at room temperature and exposed to X-ray film (Kodak XAR) in dark room, in signal intensifier screen (Hyper Cassette® from Amersham, USA) for 2 days at −80° C.

After 2 days of exposure, X-ray film was taken out in dark room and immersed in developer solution for 1 minute followed by immersion in water for 1 minute.

Finally the X-ray film was immersed in fixer solution for 2 minute followed by rinsing in water for 1-2 minutes and then air dried.

Washing Solution

### Wash I: 3×SSC+0.1% SDS

### Wash II: 0.5×SSC+0.1% SDS

### Wash III: 0.1×SSC+0.1% SDS

Developer Solution:
13.2 g of Pack A content was dissolved in 800 ml distilled water, after complete dissolving 89 g of Pack B component was added and dissolved by slow stirring. After complete dissolving volume was made upto 1000 ml and stored in amber bottle.

### Fixer Solution:

268 g of fixer was dissolved in 800 ml of distilled water by slow stirring, after complete dissolving volume was made upto 1000 ml, filtered through country filter paper and stored in amber bottle.

All the PCR positive transgenic cotton plants showed hybridization signals of varying sizes indicating the integration of transgene in the cotton genome. Some transgenic cotton plant showed integration of 201D1 DNA sequence in a single locus (single copy of 201D1 DNA) while few transgenic cotton plants showed integration of 201D1 DNA sequence in multiple locus. Hybridization signal is also seen in positive control, whereas the non-transgenic cotton plants did not show any hybridization signal.

The transgenic cotton plants (T0) with single copy of the transgene (SEQ ID NO: 2-201D1) DNA were subsequently selected for further experimental work. Seeds of the T0 plants were grown to obtain T1 to T4 generation progenies.

### A. Example 6 Biochemical Analyses of Putative Transgenic Cotton (T0) Plants by ELISA

Sandwich ELISA using EnviroLogix Quantiplate kit (EnviroLogix Inc., USA) was performed according to the manufacturer's instructions for quantitative estimation of the Cry2Ai protein (SEQ ID NO: 1) in the putative transgenic PCR positive (201D1 DNA sequence—SEQ ID NO 1) cotton plants. The positive and negative controls provided along with kit were used as reference. Second true leaf from the putative transgenic cotton plant was used for this experiment. About 30 mg of leaf tissue was homogenized in 500 μl of extraction buffer and centrifuged at 6,000 rpm at 4° C. for 7 minutes and supernatant was used for assay. Supernatant (100 μl) was loaded into anti-Cry2Ai-protein antibody pre-coated plate. The plate was covered with parafilm and incubated at room temperature (24° C.±2) for 15 minutes. Enzyme conjugate (100 μl) was added into each well. After one hour, wells were thoroughly washed with 1× wash buffer. Substrate (100 μl) was added into each well and incubated for 30 minutes. The reaction was stopped by adding the stop solution (0.1N hydrochloric acid). Optical density (O.D.) of plate was read at 450 nm using negative control as blank. Each sample was replicated twice and each well was considered as replication.

A graph was plotted between optical densities of different concentrations of calibrators (available with kit). The Cry2Ai protein (SEQ ID NO: 1) concentration was determined by plotting its O.D. value against the corresponding concentration level on graph. Concentration was calculated as described below,

$$μg of Cry 2 A protein per gram of tissue = \frac{\begin{matrix}Concentration on graph ( 500 μ l of \\ extraction buffer \text{/} mg of leaf tissue taken ) \times \\ dilution of sample extract\end{matrix}}{1000}$$

Amount of the Cry2Ai protein (SEQ ID NO: 1) present in the samples was expressed as microgram per gram of fresh leaf tissue. Out of 15 PCR positive cotton events (T0) screened by the Cry2Ai quantitative ELISA kit, all were found to be positive for expression of the Cry2Ai protein (SEQ ID NO: 1) in young transgenic cotton leaf tissue.

Examination of the ELISA results summarized in Table 1 reveals surprising and unexpected observation that most transgenic cotton plants harboring the constructs comprising the 201D1 DNA expressed the Cry2Ai protein (SEQ ID NO: 1) in the range of 10 μg/g to 20 μg/g fresh leaf tissue during vegetative stage in different generations and surprisingly stable in expression of the protein in further generations of the transgenic plants, it did not show significant variation across generations (T1-T4). While expression of the Cry1Ac protein was found to 5 μg/g to 10 μg/g fresh leaf tissue during vegetative stage in different generations and expression did not show significant variation across generations (T1-T4). Thus, the codon optimized synthetic DNA sequences encoding Cry2Ai protein shows significant enhancement in protein expression in transgenic plants over the prior art.

TABLE 1
Comparative ELISA analysis of transgenic cotton plants

| | | | | | | |
|---|---|---|---|---|---|---|
| T0 | T1 | T2 | T3 | T4 | Average | STD |

Expression of Cry2Ai protein in leaves of transgenic cotton plant comprising 201D1 DNA sequence
(SEP ID NO: 2) (ug/gm F · wt)

| | | | | | | | |
|---|---|---|---|---|---|---|---|
| Transgenic Cotton | | | | | | | |
| Plant comprising | | | | | | | |
| the DNA sequence | | | | | | | |
| as set forth in | | | | | | | |
| SEQ ID NO: 2 | | | | | | | |
| 201-1 | 13.70 | 14.42 | 17.44 | 17.49 | 15.43 | 15.70 | 1.54 |
| 201-2 | 19.99 | 13.57 | 17.36 | 15.49 | 17.08 | 16.70 | 2.13 |
| 201-3 | 17.68 | 23.14 | 19.21 | 18.21 | 18.01 | 19.25 | 2.01 |
| 201-4 | 10.66 | 16.34 | 16.57 | 15.47 | 16.36 | 15.08 | 2.24 |
| 201-5 | 8.48 | 7.69 | 7.06 | 9.30 | 9.21 | 8.35 | 0.87 |
| 201-6 | 13.05 | 16.65 | 15.78 | 17.54 | 18.65 | 16.33 | 1.89 |
| 201-7 | 17.14 | 7.51 | 7.91 | 13.50 | 7.13 | 10.64 | 4.00 |
| 201-8 | 12.55 | 11.27 | 14.90 | 13.53 | 13.31 | 13.11 | 1.19 |
| 201-9 | 13.94 | 12.20 | 12.73 | 12.80 | 12.97 | 12.93 | 0.57 |
| 201-10 | 12.56 | 12.02 | 12.23 | 12.45 | 12.53 | 12.36 | 0.21 |
| 201-11 | 12.95 | 19.16 | 18.07 | 19.16 | 19.42 | 17.75 | 2.45 |
| 201-12 | 12.13 | 11.03 | 12.38 | 12.81 | 14.23 | 12.52 | 1.04 |
| 201-13 | 11.63 | 17.95 | 16.45 | 18.59 | 17.45 | 16.41 | 2.49 |
| 201-14 | 14.04 | 12.55 | 15.37 | 14.19 | 13.31 | 13.89 | 0.94 |
| 201-15 | 16.91 | 14.80 | 13.15 | 13.15 | 15.62 | 14.72 | 1.45 |

Expression of insecticidal Cry1Ac protein in leaves of transgenic cotton plant comprising
cry1Ac gene (ug/gm F · wt)

| | | | | | | | |
|---|---|---|---|---|---|---|---|
| Transgenic Cotton | | | | | | | |
| Plant expressing | | | | | | | |
| Cry1Ac protein | | | | | | | |
| P1 | 9.09 | 5.45 | 5.63 | 5.63 | 5.63 | 6.28 | 1.40 |
| P2 | 8.77 | 8.09 | 8.77 | 8.77 | 8.77 | 8.64 | 0.27 |
| P3 | 10.45 | 7.73 | 7.15 | 7.15 | 7.15 | 7.92 | 1.28 |
| P4 | 9.90 | 4.16 | 4.95 | 4.95 | 4.95 | 5.78 | 2.08 |
| P5 | 9.19 | 8.38 | 9.19 | 9.19 | 9.19 | 9.03 | 0.32 |

Although the foregoing invention has been described in some detail by way of illustration and example for purposes of clarity of understanding, it will be obvious that certain changes and modifications may be practiced within the scope of the present disclosure.

We claim:

1. A codon optimized synthetic nucleotide sequence encoding protein having amino acid sequence as set forth in SEQ ID NO: 1, wherein said nucleotide sequence is selected from a group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and (b).

2. A nucleic acid molecule comprising a codon optimized sequence for expression in a plant selected from the group consisting of: (a) the nucleotide sequence as set forth in SEQ ID NO: 2; (b) a nucleotide sequence which specifically hybridizes to at least 10 nucleotides of the nucleotide sequence as set forth in SEQ ID NO: 2 from nucleotide position 262 to 402 and/or 1471 to 1631; and (c) a nucleotide sequence complementary to the nucleotide sequence of (a) and b).

3. The codon optimized synthetic nucleotide sequence as claimed in claim 1 or 2, wherein the nucleotide sequence which specifically hybridizes to the nucleotide sequence as set forth in SEQ ID NO: 2 is selected from the group consisting of SEQ ID NO: 3, SEQ ID NO: 4, SEQ ID NO: 5 and SEQ ID NO: 6.

4. A recombinant DNA comprising the codon optimized synthetic nucleotide sequence as claimed in claim 1, wherein the nucleotide sequence is operably linked to a heterologous regulatory element.

5. The recombinant DNA as claimed in claim 4, wherein said codon optimized synthetic nucleotide sequence optionally comprises a selectable marker gene, a reporter gene or a combination thereof.

6. The recombinant DNA as claimed in claim 4, wherein said codon optimized synthetic nucleotide sequence optionally comprises a DNA sequence encoding a targeting or transit peptide for targeting to the vacuole, mitochondrium, chloroplast, plastid, or for secretion.

7. A DNA construct for expression of an insecticidal protein in plant comprising a 5′ non-translated sequence, a coding sequence encoding an insecticidal Cry2Ai protein comprising the amino acid sequence of SEQ ID NO: 1 or an insecticidal portion thereof, and a 3′ non-translated region, wherein said 5′ non-translated sequence comprises a promoter functional in a plant cell, said coding sequence is a codon optimized synthetic nucleotide sequence as claimed in claim 1, and wherein said 3′ non-translated sequence comprises a transcription termination sequence and a polyadenylation signal.

8. A plasmid vector comprising the codon optimized synthetic nucleotide sequence as claimed in claim 1.

9. A host cell comprising the codon optimized synthetic nucleotide sequence as claimed in claim 1.

10. The host cell as claimed in claim 9, wherein said host cell is a plant, bacterial, virus, fungi or a yeast cell.

11. The host cell as claimed in claim 10, wherein said bacterial cell is Agrobacterium or E. coli.

12. A method for conferring an insect resistance in a plant comprising
(a) inserting into a plant cell a codon optimized synthetic nucleotide sequence as claimed in claim 1, wherein the nucleotide sequence is operably linked to a (i) promoter functional in a plant cell and (ii) a terminator;
(b) obtaining a transformed plant cell from the plant cell of step (a), wherein said transformed plant cell comprises the said codon optimized synthetic nucleotide sequence of claim 1; and
(c) generating a transgenic plant from said transformed plant cell of step (b), wherein said transgenic plant comprises the said codon optimized synthetic nucleotide sequence of claim 1.

13. A transgenic plant obtained by the method as claimed in claim 12.

14. A transgenic plant comprising the codon optimized synthetic nucleotide sequence as claimed in claim 1.

15. The transgenic plant as claimed in claim 13 or 14, wherein said plant is selected from a group consisting of rice, wheat, corn, sorghum, oat, millet, legume, cotton, tomato, eggplant, cabbage, cauliflower, broccoli, Brassica sp., beans, pea, pigeonpea, potato, pepper, cucurbit, lettuce, sweet potato canola, soybean, alfalfa, peanuts, sunflower, safflower, tobacco, sugarcane, cassava, coffee, pineapple, citrus, cocoa, tea, banana and melon.

16. A tissue, seed or a progeny obtained from the transgenic plant as claimed in claim 13 or 14, wherein said seed or progeny comprises the codon optimized synthetic nucleotide sequence as claimed in claim 1.

17. A biological sample derived from the tissues or seed or progeny as claimed in claim 16, wherein said sample comprising a detectable amount of said codon optimized synthetic nucleotide sequence as claimed in claim 1.

18. A commodity product derived from the transgenic plant as claimed in claim 13 or 14, wherein said product comprises a detectable amount of said codon optimized synthetic nucleotide sequence as claimed in claim 1.

19. A composition comprising Bacillus thuringiensis comprising the codon optimized synthetic nucleotide sequence as claimed in claim 1 encoding Cry2Ai protein having amino acid sequence as set forth in SEQ ID NO: 1.

20. The composition as claimed in claim 19, wherein said composition optionally comprises an additional insecticidal agent toxic to same insect pest but exhibiting a different mode of its insecticidal activity from said insecticidal protein.

21. The composition as claimed in claim 20, wherein said insecticidal agent is selected from the group consisting of a Bacillus toxin, a Xenorhabdus toxin, a Photorhabdus toxin, and a dsRNA specific for suppression of one or more essential genes in said insect pest.

22. A method of controlling insect infestation in a crop plant and providing insect resistance management, wherein said method comprising contacting said crop plant with a insecticidally effective amount of the composition as claimed in claim 19.

23. Use of the codon optimized synthetic nucleotide sequence as claimed in claim 1 for production of insect resistant transgenic plants.

24. Use of the codon optimized synthetic nucleotide sequence as claimed in claim 1 for production of insecticidal composition, wherein the composition comprises Bacillus thuringiensis cells comprising the said nucleotide sequences.
